SEQUENCE LISTING (20 sequences)

SEQ ID NO: 1 - protein, 233 aa, Staphylococcus sp
SEKSEEINEKDLRKKSELQRNALSNLRQIYYYNEKAITENKESDDQFLENTLLFKGFFTGHPWYNDLLVDLGSKDATNKYKGKKVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNKTINSENLHIDLYLYTT

SEQ ID NO: 2 - protein, 233 aa, Staphylococcus sp
SEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTS

SEQ ID NO: 3 - protein, 233 aa, Artificial Sequence
  Mutated Protein
SEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 4 - protein, 233 aa, Artificial Sequence
  Mutated Protein
SEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIAIYLYTS

SEQ ID NO: 5 - protein, 679 aa, Artificial Sequence
  Conjugated Protein
  MISC_FEATURE (460)..(679): Light Chain
QVQLQQPGAELVRPGASVKLSCKASGYTFTNYWINWVKQRPGQGLEWIGNIYPSYIYTNYNQEFKDKVTLTVDESSSTAYMQLSSPTSEDSAVYYCTRSPYGYDEYGLDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTSDIVMTQSPSSLTVTAGEKVTMNCKSSQSLLNSRNQKNYLTWYQQKPGQPPKLLIYWASTRESGVPDRFTGSGSGTDFTLTISSVQAEDLAVYYCQNDYVYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 6 - protein, 672 aa, Artificial Sequence
  Mutated and Conjugated Protein
  MISC_FEATURE (459)..(672): Light Chain
EVQLQQSGPDLVKPGASVKISCKASGYSFTGYYMHWVKQSHGKSLEWIGRINPNNGVTLYNQKFKDKAILTVDKSSTTAYMELRSLTSEDSAVYYCARSTMITNYVMDYWGQVTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIAIYLYTSSIVMTQTPTSLLVSAGDRVTITCKASQSVSNDVAWYQQKPGQSPKLLISYTSSRYAGVPDRFSGSGSGTDFTLTISSVQAEDLAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 7 - protein, 672 aa, Artificial Sequence
  Mutated and Conjugated Protein
  MISC_FEATURE (459)..(672): Light Chain
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTSIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 8 - protein, 458 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 9 - protein, 214 aa, Artificial Sequence
  Synthetic polypeptide
SIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 10 - protein, 233 aa, Artificial Sequence
  Mutated Protein
SEKSEEINEKDLRKKSELQGTALGNLKQIYYYNEKAITENKESDDQFLENTLLFKGFFTGHPWYNDLLVDLGSKDATNKYKGKKVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNKTINSENLHIALYLYTT

SEQ ID NO: 11 - protein, 5 aa, Artificial Sequence
  Synthetic polypeptide
YSWIN

SEQ ID NO: 12 - protein, 17 aa, Artificial Sequence
  Synthetic polypeptide
RIFPGDGDTDYNGKFKG

SEQ ID NO: 13 - protein, 10 aa, Artificial Sequence
  Synthetic polypeptide
NVFDGYWLVY

SEQ ID NO: 14 - protein, 16 aa, Artificial Sequence
  Synthetic polypeptide
RSSKSLLHSNGITYLY

SEQ ID NO: 15 - protein, 7 aa, Artificial Sequence
  Synthetic polypeptide
QMSNLVS

SEQ ID NO: 16 - protein, 9 aa, Artificial Sequence
  Synthetic polypeptide
AQNLELPYT

SEQ ID NO: 17 - protein, 119 aa, Artificial Sequence
  Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 18 - protein, 115 aa, Artificial Sequence
  Synthetic polypeptide
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 19 - protein, 449 aa, Artificial Sequence
  Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 219 aa, Artificial Sequence
  Synthetic polypeptide
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0285588 A1
(43) Pub. Date: Sep. 14, 2023

(54) SUPERANTIGEN CONJUGATE FOR USE IN METHODS AND COMPOSITIONS FOR TREATING CANCER

(71) Applicant: NeoTX Therapeutics Ltd., Rehovot (IL)

(72) Inventors: Michal Shahar, Rishon Lezion (IL); Meir Azulay, Givatayim (IL); Asher Nathan, Rishon LeZion (IL)

(21) Appl. No.: 18/006,243

(22) PCT Filed: Jul. 20, 2021
(86) PCT No.: PCT/IL2021/050882

§ 371 Date: Jan. 20, 2023

Related U.S. Application Data
(60) Provisional application No. 63/053,859, filed on Jul. 20, 2020.

(51) Int. Cl. A61K 47/68; A61P 35/00; A61K 39/395; A61K 39/085; A61K 31/397

(52) U.S. Cl. CPC A61K 47/6851; A61P 35/00; A61K 39/3955; A61K 39/085; A61K 31/397; A61K 2039/6031

(57) ABSTRACT

The invention provides methods or compositions for treating cancer using a superantigen conjugate in combination with a B-cell depleting agent, e.g., an anti-CD20 antibody.

### FIELD OF THE INVENTION

The invention relates generally to superantigen conjugate and its use in therapy.

### BACKGROUND

According to the American Cancer Society, more than one million people in the United States are diagnosed with cancer each year. Cancer is a disease that results from uncontrolled proliferation of cells that were once subject to natural control mechanisms but have been transformed into cancerous cells that continue to proliferate in an uncontrolled manner.

In recent years, a number of immunotherapies have been developed that have attempted to harness the subject's immune system to find and destroy cancer cells. Such immunotherapies include, for example, those that are designed to boost the body's natural defenses for fighting cancer using natural molecules made by the body, or alternatively, through administration of recombinant molecules designed to improve, better target, or restore immune system function. Certain immunotherapies include the administration of compounds known to be general immune system enhancers, such as cytokines, for example, IL-2 and interferon. While various immunotherapies developed to date have shown efficacy, they can be associated with side effects including, for example, off-target activities, allergic reactions to the active agents administered including the potential for cytokine storms, a loss of potency caused by the stimulation of antibodies that bind and neutralize the active agents, a decrease in blood cell number, and fatigue.

Other immunotherapies utilize molecules referred to as immune checkpoint inhibitors, which enhance immune responses to cancer. Such checkpoint inhibitors function to inhibit the ability of cancer cells to block immune inhibitory checkpoints thereby resulting in an enhancement of potency of an anti-cancer therapy. A first-generation immune checkpoint inhibitor ipilimumab (YERVOY®; Bristol-Myers Squibb) was approved by the U.S. Food and Drug Administration in 2011 and is an IgG1 monoclonal antibody that can cause ADCC-mediated regulatory T-cell (Treg) cytotoxicity. More recently, PD-1 inhibitors have been approved such as nivolumab and pembrolizumab, which prevent the inhibitory signals between PD-1 and PD-L1. While these drugs have potentiated durable responses in some patients, the response rates of these drugs as monotherapy have been low and in the range of 21%, and the complete response rate has been about 1% in several studies.

Accordingly, despite the significant developments that have been made in the fields of immunotherapy and oncology, there is still a need for safe and effective immunotherapies for treating cancer.

### SUMMARY OF THE INVENTION

The invention is based, in part, upon the discovery that a targeted immune response against a cancer in a subject can be significantly enhanced by combining a superantigen conjugate comprising a superantigen covalently linked to a targeting moiety that binds a cancer antigen with a B-cell depleting agent (e.g., an anti-CD20 antibody).

Furthermore, it has been discovered that administration of a superantigen conjugate comprising a superantigen covalently linked to a targeting moiety that binds a cancer antigen to a subject in combination with a B-cell depleting agent (e.g., an anti-CD20 antibody) can increase the number of T-cells in a tumor in the subject (i.e., increase T-cell tumor infiltration), increase the ratio of CD8+ T-cells to CD4+ T-cells in a tumor in the subject, and/or decrease the number of regulatory T-cells (Tregs) in a tumor in the subject.

Accordingly, in one aspect, the invention provides a superantigen conjugate for use in treating cancer, said superantigen being covalently linked to a targeting moiety that binds a cancer antigen wherein said use is in combination with a B-cell depleting agent, and said B-cell depleting agent is administered to a subject in need before administration of the superantigen conjugate to the subject. In certain embodiments, the cancer comprises a tumor, e.g., a solid tumor.

In accordance with a further aspect, the invention provides a superantigen conjugate for use in promoting infiltration of T-cells into a tumor, said superantigen being covalently linked to a targeting moiety that binds a cancer antigen expressed by cancerous cells, wherein said use is in combination with a B-cell depleting agent; and said B-cell depleting agent is administered to a subject in need before administration of the superantigen conjugate to the subject.

Further provided, in accordance with some aspects, is a superantigen conjugate for use in increasing the ratio of CD8+ T-cells to CD4+ T-cells in a tumor, said superantigen being covalently linked to a targeting moiety that binds a cancer antigen expressed by cancerous cells, wherein said use is in combination with B-cell depleting agent and said B-cell depleting agent is administered to a subject in need before administration of the superantigen conjugate to the subject.

Yet, a further aspect of the invention provides a superantigen conjugate for use in reducing infiltration of regulatory T-cells (Tregs) into a tumor, said superantigen being covalently linked to a targeting moiety that binds a cancer antigen expressed by cancerous cells, wherein said use is in combination with B-cell depleting agent and said B-cell depleting agent is administered to a subject in need before administration of the superantigen conjugate to the subject.

The invention also provides, in accordance with a further aspect, a superantigen conjugate for use in treating cancer, said superantigen being covalently linked to a targeting moiety that binds a cancer antigen expressed by cancerous cells, wherein said use is in combination with B-cell depleting agent and said B-cell depleting agent is administered to a subject in need before administration of the superantigen conjugate to the subject.

In accordance with a further aspect, the invention provides a method of improving the efficacy of a superantigen conjugate comprising a superantigen covalently linked to a targeting moiety that binds a cancer antigen in treating cancer in a subject in need thereof. The method comprises administering to the subject an effective amount of a B-cell depleting agent in combination with the superantigen conjugate. In certain embodiments, the method comprises administering to the subject an effective amount of the B-cell depleting agent prior to administration of the superantigen conjugate to the subject. In certain embodiments, the cancer comprises a tumor, e.g., a solid tumor.

In another aspect, the invention provides a method of promoting infiltration of T-cells (e.g., CD8+ T-cells) into a tumor in a subject in need thereof. The method comprises administering to the subject: (i) an effective amount of a B-cell depleting agent; and (ii) an effective amount of a superantigen conjugate comprising a superantigen covalently linked to a targeting moiety that binds a cancer antigen expressed by cancerous cells of the tumor, thereby to promote infiltration of T-cells into the tumor. In certain embodiments, the method comprises first administering to the subject the B-cell depleting agent, and then administering to the subject the superantigen conjugate.

In another aspect, the invention provides a method of increasing the ratio of CD8+ T-cells to CD4+ T-cells in a tumor in a subject in need thereof. The method comprises administering to the subject: (i) an effective amount of a B-cell depleting agent; and (ii) an effective amount of a superantigen conjugate comprising a superantigen covalently linked to a targeting moiety that binds a cancer antigen expressed by cancerous cells of the tumor, thereby to increase the ratio of CD8+ T-cells to CD4+ T-cells in the tumor. In certain embodiments, the method comprises first administering to the subject the B-cell depleting agent, and then administering to the subject the superantigen conjugate.

In another aspect, the invention provides a method of reducing infiltration of regulatory T-cells (Tregs) into a tumor in a subject in need thereof. The method comprises administering to the subject: (i) an effective amount of a B-cell depleting agent; and (ii) an effective amount of a superantigen conjugate comprising a superantigen covalently linked to a targeting moiety that binds a cancer antigen expressed by cancerous cells of the tumor, thereby to reduce infiltration of Tregs into the tumor. In certain embodiments, the method comprises first administering to the subject the B-cell depleting agent, and then administering to the subject the superantigen conjugate.

In another aspect, the invention provides a method of treating cancer in a subject in need thereof. The method comprises administering to the subject: (i) an effective amount of a B-cell depleting agent; and (ii) an effective amount of a superantigen conjugate comprising a superantigen covalently linked to a targeting moiety that binds a cancer antigen expressed by cancerous cells of the tumor. In certain embodiments, the method comprises first administering to the subject the B-cell depleting agent, and then administering to the subject the superantigen conjugate. In certain embodiments, the cancer comprises a tumor, e.g., a solid tumor.

In each of the foregoing aspects, administration of the B-cell depleting agent and the superantigen conjugate to the subject (i) promotes infiltration of T-cells into the tumor, (ii) increases the ratio of CD8+ T-cells to CD4+ T-cells in the tumor, and/or (iii) reduces infiltration of Tregs into the tumor.

Furthermore, in each of the foregoing aspects, the superantigen can comprise Staphylococcal enterotoxin A or an immunologically variant and/or fragment thereof. For example, the superantigen can comprise the amino acid sequence of SEQ ID NO: 3, or an immunologically reactive variant and/or fragment thereof.

Furthermore, in each of the foregoing aspects, the cancer antigen is selected from EpCAM and 5T4. In certain embodiments of the any of the foregoing methods, the targeting moiety is an antibody, e.g., an anti-5T4 antibody or an anti-EpCAM antibody. In certain embodiments, the targeting moiety is an anti-5T4 antibody, e.g., an anti-5T4 antibody comprising a Fab fragment that binds a 5T4 cancer antigen. In certain embodiments, the anti-5T4 antibody comprises a heavy chain comprising amino acid residues 1-222 of SEQ ID NO: 8 and a light chain comprising amino acid residues 1-214 of SEQ ID NO: 9. In certain embodiments, the superantigen conjugate comprises a first protein chain comprising SEQ ID NO: 8 and a second protein chain comprising SEQ ID NO: 9.

In certain embodiments of any of the foregoing aspects, the B-cell depleting agent is an anti-CD20 antibody, e.g., an anti-CD20 antibody selected from ibritumomab, obinutuzumab, ocaratuzumab, ocrelizumab, ofatumumab, rituximab, veltuzumab, tositumomab, ublituximab, veltuzumab, PRO131921, and TRU-015. In certain embodiments, the anti-CD20 antibody is obinutuzumab.

In certain embodiments of any of the foregoing aspects, the cancer is a 5T4- or an EpCAM-expressing cancer. The cancer can be selected from breast cancer, bladder cancer, cervical cancer, colon cancer, colorectal cancer, endometrial cancer, gastric cancer, head and neck cancer, liver cancer, melanoma, mesothelioma, non-small cell lung cancer, ovarian cancer, pancreatic cancer, prostate cancer, renal cell cancer, and skin cancer. In certain embodiments, the cancer is selected from colon cancer and colorectal cancer. In certain embodiments, the cancer is a hematopoietic cancer.

In each of the foregoing aspects, the combination or method can further comprise administering an immunopotentiator to the subject. Exemplary immunopotentiators include a CTLA-4-based inhibitor or a PD-1-based inhibitor, e.g., a PD-1 or PD-L1 inhibitor. In certain embodiments, the PD-1 inhibitor is an anti-PD-1 antibody, e.g., an anti-PD-1 antibody selected from nivolumab, pembrolizumab, and cemiplimab. In certain embodiments, the PD-L1 inhibitor is an anti-PD-L1 antibody, e.g., an anti-PD-L1 antibody selected from atezolizumab, avelumab, and durvalumab.

In each of the foregoing aspects, the combination or method can further comprise administering a chemotherapeutic agent, e.g., docetaxel, to the subject.

In another aspect, the invention provides a pharmaceutical composition comprising: (i) an effective amount of a superantigen conjugate comprising a superantigen covalently linked to a targeting moiety that binds a cancer antigen expressed by cancerous cells within the subject; (ii) an effective amount of a B-cell depleting agent; and (iii) a pharmaceutically acceptable excipient.

These and other aspects and features of the invention are described in the following detailed description, figures, and claims.

### BRIEF DESCRIPTION OF THE DRAWINGS

The foregoing and other objects, features and advantages of the invention will become apparent from the following description of preferred embodiments, as illustrated in the accompanying drawings. Like referenced elements identify common features in the corresponding drawings. The drawings are not necessarily to scale, with emphasis instead being placed on illustrating the principles of the present invention.

FIG. 1 is a sequence alignment showing the homologous A-E regions in certain exemplary wild type and modified superantigens.

FIG. 2 is an amino acid sequence corresponding to an exemplary superantigen conjugate, naptumomab estafenatox, which comprises two protein chains. The first protein chain comprises residues 1 to 458 of SEQ ID NO: 7 (see also, SEQ ID NO: 8), and includes a chimeric 5T4 Fab heavy chain, corresponding to residues of SEQ ID NO: 7, and the SEA/E-120 superantigen, corresponding to residues 226 to 458 of SEQ ID NO: 7, covalently linked via a GGP tripeptide linker, corresponding to residues 223-225 of SEQ ID NO: 7. The second chain comprises residues 459 to 672 of SEQ ID NO: 7 (see also, SEQ ID NO: 9) and includes a chimeric 5T4 Fab light chain. The two protein chains are held together by non-covalent interactions between the Fab heavy and light chains.

FIG. 3 is a dot plot showing staining for B-cells in spleens of mice injected with a single 250 μg/mouse dose of either rat IgG2b isotype or anti-mouse CD20 antibody. Staining was performed 14 days post injection (day 7 as described in Example 1). For each group, the percentage of cells with the B220/CD45R B-cell surrogate marker out of total lymphocytes is depicted. FIG. 3A shows results for the rat IgG2b isotype group (“control”), and FIG. 3B shows results for the anti-mouse CD20 antibody group (“B cell depleted”).

FIG. 4 is a line graph illustrating MC38-EpCAM tumor volume following treatment with tumor targeted superantigen (“TTS”), tumor targeted superantigen in combination with anti-CD20 antibody (“B cell depletion TTS”), anti-CD20 antibody (“B cell depletion control”), or IgG control (“Control”). The mean tumor volume of 10 mice/group±SE is depicted. **p=0.0013; ***p=0.0008; ****p<0.0001.

FIG. 5 is a line graph illustrating MC38-EpCAM tumor volume following treatment with anti-PD-L1 antibody (“PD-L1”), anti-PD-L1 antibody in combination with anti-CD20 antibody (“B cell depletion PD-L1”), anti-CD20 antibody (“B cell depletion control”), or IgG control (“Control”). The mean tumor volume of 10 mice/group±SE is depicted. ****p<0.0001.

FIGS. 6A-6D are graphs illustrating the infiltration of T-cell subsets into tumors excised from control, tumor targeted superantigen (“TTS”), and anti-PD-L1 antibody (“anti-PD-L1”) treated mice with (“B depleted”) or without (“non-depleted”) B-cell depletion by anti-CD20 antibody treatment. FIG. 6A depicts general CD8+ T-cell infiltration. FIG. 6B depicts the ratio of general CD8+ T-cells to CD4+T-cells in the tumors. FIG. 6C depicts Vβ3+ CD8+ T-cell infiltration. FIG. 6D depicts the ratio of Vβ3+ CD8+ T-cells to CD4+ T-cells in the tumors.

### DETAILED DESCRIPTION

The invention is based, in part, upon the discovery that a targeted immune response against a cancer in a subject can be significantly enhanced by combining a superantigen conjugate comprising a superantigen covalently linked to a targeting moiety that binds a cancer antigen with a B-cell depleting agent (e.g., an anti-CD20 antibody)

Furthermore, it has been discovered that administration of a superantigen conjugate comprising a superantigen covalently linked to a targeting moiety that binds a cancer antigen to a subject in combination with a B-cell depleting agent (e.g., an anti-CD20 antibody) can increase the number of T-cells in a tumor in the subject (i.e., increase T-cell tumor infiltration), increase the ratio of CD8+ T-cells to CD4+ T-cells in a tumor in the subject, and/or decrease the number of regulatory T-cells (Tregs) in a tumor in the subject.

Accordingly, the invention provides a superantigen conjugate for use in treating cancer, said superantigen being covalently linked to a targeting moiety that binds a cancer antigen wherein said use is in combination with a B-cell depleting agent, and said B-cell depleting agent is administered to a subject in need before administration of the superantigen conjugate to the subject. In certain embodiments, the cancer comprises a tumor, e.g., a solid tumor.

In accordance with a further aspect, the invention provides a superantigen conjugate for use in promoting infiltration of T-cells into a tumor, said superantigen being covalently linked to a targeting moiety that binds a cancer antigen expressed by cancerous cells, wherein said use is in combination with a B-cell depleting agent; and said B-cell depleting agent is administered to a subject in need before administration of the superantigen conjugate to the subject.

Further provided, in accordance with some aspects, is a superantigen conjugate for use in increasing the ratio of CD8+ T-cells to CD4+ T-cells in a tumor, said superantigen being covalently linked to a targeting moiety that binds a cancer antigen expressed by cancerous cells, wherein said use is in combination with B-cell depleting agent and said B-cell depleting agent is administered to a subject in need before administration of the superantigen conjugate to the subject.

Yet, a further aspect of the invention provides a superantigen conjugate for use in reducing infiltration of regulatory T-cells (Tregs) into a tumor, said superantigen being covalently linked to a targeting moiety that binds a cancer antigen expressed by cancerous cells, wherein said use is in combination with B-cell depleting agent and said B-cell depleting agent is administered to a subject in need before administration of the superantigen conjugate to the subject.

The invention also provides, in accordance with a further aspect, a superantigen conjugate for use in treating cancer, said superantigen being covalently linked to a targeting moiety that binds a cancer antigen expressed by cancerous cells, wherein said use is in combination with B-cell depleting agent and said B-cell depleting agent is administered to a subject in need before administration of the superantigen conjugate to the subject.

In yet a further aspect, the invention provides a method of improving the efficacy of a superantigen conjugate comprising a superantigen covalently linked to a targeting moiety that binds a cancer antigen in treating cancer in a subject in need thereof. The method comprises administering to the subject an effective amount of a B-cell depleting agent in combination with the superantigen conjugate. In certain embodiments, the method comprises administering to the subject an effective amount of the B-cell depleting agent prior to administration of the superantigen conjugate to the subject.

In another aspect, the invention provides a method of promoting infiltration of T-cells (e.g., CD8+ T-cells) into a tumor in a subject in need thereof. The method comprises administering to the subject: (i) an effective amount of a B-cell depleting agent; and (ii) an effective amount of a superantigen conjugate comprising a superantigen covalently linked to a targeting moiety that binds a cancer antigen expressed by cancerous cells of the tumor, thereby to promote infiltration of T-cells into the tumor. In certain embodiments, the method comprises first administering to the subject the B-cell depleting agent, and then administering to the subject the superantigen conjugate.

In another aspect, the invention provides a method of increasing the ratio of CD8+ T-cells to CD4+ T-cells in a tumor in a subject in need thereof. The method comprises administering to the subject: (i) an effective amount of a B-cell depleting agent; and (ii) an effective amount of a superantigen conjugate comprising a superantigen covalently linked to a targeting moiety that binds a cancer antigen expressed by cancerous cells of the tumor, thereby to increase the ratio of CD8+ T-cells to CD4+ T-cells in the tumor. In certain embodiments, the method comprises first administering to the subject the B-cell depleting agent, and then administering to the subject the superantigen conjugate.

In another aspect, the invention provides a method of reducing infiltration of regulatory T-cells (Tregs) into a tumor in a subject in need thereof. The method comprises administering to the subject: (i) an effective amount of a B-cell depleting agent; and (ii) an effective amount of a superantigen conjugate comprising a superantigen covalently linked to a targeting moiety that binds a cancer antigen expressed by cancerous cells of the tumor, thereby to reduce infiltration of Tregs into the tumor. In certain embodiments, the method comprises first administering to the subject the B-cell depleting agent, and then administering to the subject the superantigen conjugate.

In another aspect, the invention provides a method of treating cancer in a subject in need thereof. The method comprises administering to the subject: (i) an effective amount of a B-cell depleting agent; and (ii) an effective amount of a superantigen conjugate comprising a superantigen covalently linked to a targeting moiety that binds a cancer antigen expressed by cancerous cells of the tumor. In certain embodiments, the method comprises first administering to the subject the B-cell depleting agent, and then administering to the subject the superantigen conjugate.

In another aspect, the invention provides a pharmaceutical composition comprising: (i) an effective amount of a superantigen conjugate comprising a superantigen covalently linked to a targeting moiety that binds a cancer antigen expressed by cancerous cells within the subject; (ii) an effective amount of a B-cell depleting agent; and (iii) a pharmaceutically acceptable excipient.

### I. Definitions

Unless defined otherwise, technical and scientific terms used herein have the same meaning as commonly understood by one of ordinary skill in the art to which this invention belongs. For purposes of the present invention, the following terms are defined below.

As used herein, the terms “a” or “an” may mean one or more. For example, a statement such as “treatment with a superantigen conjugate and a B-cell depleting agent,” can mean treatment: with one superantigen conjugate and B-cell depleting agent; with more than one superantigen conjugate and one B-cell depleting agent; with one superantigen conjugate and more than one B-cell depleting agent; or with more than one superantigen conjugate and more than one B-cell depleting agent.

As used herein, unless otherwise indicated, the term “antibody” is understood to mean an intact antibody (e.g., an intact monoclonal antibody) or antigen-binding fragment of an antibody, including an intact antibody or antigen-binding fragment of an antibody (e.g., a phage display antibody including a fully human antibody, a semisynthetic antibody or a fully synthetic antibody) that has been optimized, engineered or chemically conjugated. Examples of antibodies that have been optimized are affinity-matured antibodies. Examples of antibodies that have been engineered are Fc optimized antibodies, antibodies engineered to reduce immunogenicity, and multi-specific antibodies (e.g., bispecific antibodies). Examples of antigen-binding fragments include Fab, Fab′, F(ab′)₂, Fv, single chain antibodies (e.g., scFv), minibodies and diabodies. An antibody conjugated to a toxin moiety is an example of a chemically conjugated antibody. In certain embodiments, an antibody has a human IgG1, IgG2, IgG3, IgG4, or IgE isotype. In certain embodiments, an antibody binds a target antigen (e.g., CD20) with an affinity (Kd) of at least 100 nM, 50 nM, 20 nM, 15 nM, 10 nM, 9 nM, 8 nM, 7 nM, 6 nM, 5 nM, 4 nM, 3 nM, 2 nM, 1 nM, 0.1 nM, 0.01 nM, or 0.001 nM, as measured by surface plasmon resonance or bio-layer interferometry.

As used herein, the terms “cancer” and “cancerous” are understood to mean the physiological condition in mammals that is typically characterized by unregulated cell growth. Examples of cancer include, but are not limited to, melanoma, carcinoma, lymphoma, blastoma, sarcoma, and leukemia or lymphoid malignancies. More particular examples of cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, bone cancer, brain cancer, retinoblastoma, endometrial cancer or uterine carcinoma, salivary gland carcinoma, kidney or renal cancer, prostate cancer, vulval cancer, thyroid cancer, hepatic carcinoma, anal carcinoma, penile carcinoma, testicular cancer, as well as head and neck cancer, gum or tongue cancer. The cancer comprises cancer or cancerous cells, for example, the cancer may comprise a plurality of individual cancer or cancerous cells, for example, a leukemia, or a tumor comprising a plurality of associated cancer or cancerous cells.

As used herein, the term “refractory” refers to a cancer that does not respond or no longer responds to a treatment. In certain embodiments, a refractory cancer can be resistant to a treatment before or at the beginning of the treatment. In other embodiments, the refractory cancer can become resistant during or after a treatment. A refractory cancer is also called a resistant cancer. As used herein, the term “recurrence” or “relapse” refers to the return of a refractory cancer or the signs and symptoms of a refractory cancer after a positive response a prior treatment (e.g., a reduction in tumor burden, a reduction in tumor volume, a reduction in tumor metastasis, or a modulation of a biomarker indicative of a positive response to a treatment).

As used herein, the term “immunogen” is a molecule that provokes (evokes, induces, or causes) an immune response. This immune response may involve antibody production, the activation of certain cells, such as, for example, specific immunologically-competent cells, or both. An immunogen may be derived from many types of substances, such as, but not limited to, molecules from organisms, such as, for example, proteins, subunits of proteins, killed or inactivated whole cells or lysates, synthetic molecules, and a wide variety of other agents both biological and nonbiological. It is understood that essentially any macromolecule (including naturally occurring macromolecules or macromolecules produced via recombinant DNA approaches), including virtually all proteins, can serve as immunogens.

As used herein, the term “immunogenicity” relates to the ability of an immunogen to provoke (evoke, induce, or cause) an immune response. Different molecules may have differing degrees of immunogenicity, and a molecule having an immunogenicity that is greater compared to another molecule is known, for example, to be capable of provoking (evoking, inducing, or causing) a greater immune response than would an agent having a lower immunogenicity.

As used herein, the term “antigen” as used herein refers to a molecule that is recognized by antibodies, specific immunologically-competent cells, or both. An antigen may be derived from many types of substances, such as, but not limited to, molecules from organisms, such as, for example, proteins, subunits of proteins, nucleic acids, lipids, killed or inactivated whole cells or lysates, synthetic molecules, and a wide variety of other agents both biological and non-biological.

As used herein, the term “antigenicity” relates to the ability of an antigen to be recognized by antibodies, specific immunologically-competent cells, or both.

As used herein, the term “epitope spreading” refers to the diversification of the epitope specificity of an immune response from an initial epitope-specific immune response directed against an antigen to other epitopes on that antigen (intramolecular spreading) or other antigens (intermolecular spreading). Epitope spreading allows a subject's immune system to determine additional target epitopes not initially recognized by the immune system in response to the original therapeutic protocol while reducing the possibility of escape variants in a tumor population and thus affect progression of disease.

As used herein, the term “immune response” refers to a response by a cell of the immune system, such as a B-cell, T-cell (CD4+ or CD8+), regulatory T-cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4+ T-cell, CD8+ T-cell, or B-cell via their antigen-specific receptor. In some embodiments, an immune response is a T-cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response.

As used herein, the term “major histocompatibility complex,” or “MHC,” refers to a specific cluster of genes, many of which encode evolutionarily related cell surface proteins involved in antigen presentation, that are important determinants of histocompatibility. Class I MEW, or MHC-I, function mainly in antigen presentation to CD8⁺ T lymphocytes (CD8⁺ T-Cells). Class II MEW, or MHC-II, function mainly in antigen presentation to CD4⁺ T lymphocytes (CD4⁺T-Cells).

As used herein, the term “derived,” for example “derived from,” includes, but is not limited to, for example, wild-type molecules derived from biological hosts such as bacteria, viruses and eukaryotic cells and organisms, and modified molecules, for example, modified by chemical means or produced in recombinant expression systems.

As used herein, the terms “seroreactive,” “seroreaction” or “seroreactivity” are understood to mean the ability of an agent, such as a molecule, to react with antibodies in the serum of a mammal, such as, but not limited to, a human. This includes reactions with all types of antibodies, including, for example, antibodies specific for the molecule and nonspecific antibodies that bind to the molecule, regardless of whether the antibodies inactivate or neutralize the agent. As is known in the art, different agents may have different seroreactivity relative to one another, wherein an agent having a seroreactivity lower than another would, for example, react with fewer antibodies and/or have a lower affinity and/or avidity to antibodies than would an agent having a higher seroreactivity. This may also include the ability of the agent to elicit an antibody immune response in an animal, such as a mammal, such as a human.

As used herein, the terms “soluble T-cell receptor,” or “soluble TCR,” are understood to mean a “soluble” T-cell receptor comprising the chains of a full-length (e.g., membrane bound) receptor, except that the transmembrane region of the receptor chains are deleted or mutated so that the receptor, when expressed by a cell, will not insert into, traverse or otherwise associate with the membrane. A soluble T-cell receptor may comprise only the extracellular domains or extracellular fragments of the domains of the wild-type receptor (e.g., lacks the transmembrane and cytoplasmic domains).

As used herein, the term “superantigen” is understood to mean a class of molecules that stimulate a subset of T-cells by binding to MHC class II molecules and VP domains of T-cell receptors, thereby activating T-cells expressing particular VP gene segments. The term includes wild-type, naturally occurring superantigens, for example, those isolated from certain bacteria or expressed from unmodified genes from same, as well as modified superantigens, wherein, for example, the DNA sequence encoding a superantigen has been modified, for example, by genetic engineering, to, for example, produce a fusion protein with a targeting moiety, and/or alter certain properties of the superantigen, such as, but not limited to, its MHC class II binding (for example, to reduce affinity) and/or its seroreactivity, and/or its immunogenicity, and/or antigenicity (for example, to reduce its seroreactivity). The definition includes wild-type and modified superantigens and any immunologically reactive variants and/or fragments thereof described herein or in the following U.S. patents and patent applications: U.S. Pat. Nos. 5,858,363, 6,197,299, 6,514,498, 6,713,284, 6,692,746, 6,632,640, 6,632,441, 6,447,777, 6,399,332, 6,340,461, 6,338,845, 6,251,385, 6,221,351, 6,180,097, 6,126,945, 6,042,837, 6,713,284, 6,632,640, 6,632,441, 5,859,207, 5,728,388, 5,545,716, 5,519,114, 6,926,694, 7,125,554, 7,226,595, 7,226,601, 7,094,603, 7,087,235, 6,835,818, 7,198,398, 6,774,218, 6,913,755, 6,969,616, and 6,713,284, U.S. Patent Application Nos. 2003/0157113, 2003/0124142, 2002/0177551, 2002/0141981, 2002/0115190, 2002/0051765, and 2001/0046501, and International (PCT) Publication No. WO/03/094846.

As used herein, the term “targeting moiety” refers to any structure, molecule or moiety that is able to bind to a cellular molecule, for example, a cell surface molecule, preferably a disease specific molecule such as an antigen expressed preferentially on a cancer (or cancerous) cell. Exemplary targeting moieties include, but are not limited to, antibodies (including antigen binding fragments thereof) and the like, soluble T-cell receptors, interleukins, hormones, and growth factors.

As used herein, the terms “tumor-targeted superantigen” or “TTS” or “cancer-targeted superantigen” are understood to mean a molecule comprising one or more superantigens covalently linked (either directly or indirectly) with one or more targeting moieties.

As used herein, the term “T-cell receptor” is understood to mean a receptor that is specific to T-cells, and includes the understanding of the term as known in the art. The term also includes, for example, a receptor that comprises a disulfide-linked heterodimer of the highly variable α or β chains expressed at the cell membrane as a complex with the invariant CD3 chains, and a receptor made up of variable γ and δ chains expressed at the cell membrane as a complex with CD3 on a subset of T-cells.

As used herein, the terms “therapeutically effective amount” and “effective amount,” are understood to mean an amount of an active agent, for example, a pharmaceutically active agent or a pharmaceutical composition that produces at least some effect in treating a disease or a condition. The effective amount of pharmaceutically active agent(s) used to practice the present invention for a therapeutic treatment varies depending upon the manner of administration, the age, body weight, and general health of the subject. These terms include, but are not limited to synergistic situations such as those described in the instant invention wherein a single agent alone, such as a superantigen conjugate or a B-cell depleting agent (for example, an anti-CD20 antibody), may act weakly or not at all, but when combined with each other, for example, but not limited to, via sequential dosage, the two or more agents act to produce a synergistic result.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

As used herein, the terms “treat,” “treating” and “treatment” are understood to mean the treatment of a disease in a mammal, e.g., in a human. This includes: (a) inhibiting the disease, i.e., arresting its development; and (b) relieving the disease, i.e., causing regression of the disease state; and (c) curing the disease. As used in the context of a therapeutic treatment, the terms “prevent” or “block” are understood to completely prevent or block, or not completely prevent or block (e.g., partially prevent or block) a given act, action, activity, or event.

As used herein, the term “inhibits the growth of a cancer” is understood to mean a measurably slowing, stopping, or reversing the growth rate of the cancer or cancerous cells in vitro or in vivo. Desirably, the growth rate is slowed by 20%, 30%, 50%, or 70% or more, as determined using a suitable assay for determination of cell growth rates. Typically, a reversal of growth rate is accomplished by initiating or accelerating necrotic or apoptotic mechanisms of cell death in neoplastic cells, resulting in a shrinkage of a neoplasm.

As used herein, the terms “variant,” “variants,” “modified,” “altered,” “mutated,” and the like, are understood to mean proteins or peptides and/or other agents and/or compounds that differ from a reference protein, peptide or other compound. Variants in this sense are described below and elsewhere in greater detail. For example, changes in a nucleic acid sequence of the variant may be silent, e.g., they may not alter the amino acids encoded by the nucleic acid sequence. Where alterations are limited to silent changes of this type a variant will encode a peptide with the same amino acid sequence as the reference peptide. Changes in the nucleic acid sequence of the variant may alter the amino acid sequence of a peptide encoded by the reference nucleic acid sequence. Such nucleic acid changes may result in amino acid substitutions, additions, deletions, fusions and/or truncations in the protein or peptide encoded by the reference sequence, as discussed below. Generally, differences in amino acid sequences are limited so that the sequences of the reference and the variant are similar overall and, in many regions, identical. A variant and reference protein or peptide may differ in amino acid sequence by one or more substitutions, additions, deletions, fusions and/or truncations, which may be present in any combination. A variant may also be a fragment of a protein or peptide of the invention that differs from a reference protein or peptide sequence by being shorter than the reference sequence, such as by a terminal or internal deletion. Another variant of a protein or peptide of the invention also includes a protein or peptide which retains essentially the same function or activity as the reference protein or peptide. A variant may also be: (i) one in which one or more of the amino acid residues are substituted with a conserved or non-conserved amino acid residue and such substituted amino acid residue may or may not be one encoded by the genetic code, or (ii) one in which one or more of the amino acid residues includes a substituent group, or (iii) one in which the mature protein or peptide is fused with another compound, such as a compound to increase the half-life of the protein or peptide (for example, polyethylene glycol), or (iv) one in which the additional amino acids are fused to the mature protein or peptide, such as a leader or secretory sequence or a sequence which is employed for purification of the mature protein or peptide. Variants may be made by mutagenesis techniques, and/or altering mechanisms such as chemical alterations, fusions, adjuncts and the like, including those applied to nucleic acids, amino acids, cells or organisms, and/or may be made by recombinant means.

As used herein, the term “sequential dosage” and related terminology refers to the administration of at least a first agent (e.g., a superantigen conjugate) with at least a second agent (e.g., a B-cell depleting agent) and includes staggered doses of these agents (i.e., time-staggered) and variations in dosage amounts. This includes one agent being administered before, overlapping with (partially or totally), or after administration of another agent. This term generally considers the best administration scheme to achieve a synergistic combination of the first agent (e.g., the superantigen conjugate) and the second agent (e.g., the B-cell depleting agent). By such a dosing strategy (e.g., a sequential dosage), one may be able to achieve synergistic effects (e.g., synergistic effects of combined superantigen conjugate and B-cell depleting agent administration). In addition, the term “sequential dosage” and related terminology also includes the administration of at least a first agent (e.g., a superantigen conjugate), a second agent (e.g., a B-cell depleting agent), and more or more optional additional agents or compounds such as, for example, a corticosteroid, an immune modulator, or an agent designed to reduce potential immunoreactivity to the superantigen conjugate administered to the subject.

As used herein, the terms “systemic” and “systemically” in the context of administration are understood to mean administration of an agent such that the agent is exposed to at least one system associated with the whole body, such as but not limited to the circulatory system, immune system, and lymphatic system, rather than only to a localized part of the body, such as but not limited to within a tumor. Thus, for example, a systemic therapy or an agent administered systematically is a therapy or an agent in which at least one system associated with the entire body is exposed to the therapy or agent, as opposed to, rather than just a target tissue.

As used herein, the term “parenteral administration” includes any form of administration in which the compound is absorbed into the subject without involving absorption via the intestines. Exemplary parenteral administrations that are used in the present invention include, but are not limited to intramuscular, intravenous, intraperitoneal, or intraarticular administration.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

At various places in the present specification, values are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing and method steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### II. Superantigen Conjugate

A. Superantigens

Superantigens are bacterial proteins, viral proteins, and human-engineered proteins, capable of activating T lymphocytes, for example, at picomolar concentrations. Superantigens can also activate large subsets of T lymphocytes (T-cells). Superantigens can bind to the major histocompatibility complex I (MHCI) without being processed and, in particular, can bind to conserved regions outside the antigen-binding groove on MHC class II molecules, avoiding most of the polymorphism in the conventional peptide-binding site. Superantigens can also bind to the VP chain of the T-cell receptor (TCR) rather than binding to the hypervariable loops of the T-cell receptor. Examples of bacterial superantigens include, but are not limited to, Staphylococcal enterotoxin (SE), Streptococcus pyogenes exotoxin (SPE), Staphylococcus aureus toxic shock-syndrome toxin (TSST-1), Streptococcal mitogenic exotoxin (SME), Streptococcal superantigen (SSA), Staphylococcal enterotoxin A (SEA), Staphylococcal enterotoxin A (SEB), and Staphylococcal enterotoxin E (SEE).

The polynucleotide sequences encoding many superantigens have been isolated and cloned and superantigens expressed from these or modified (reengineered) polynucleotide sequences have been used in anti-cancer therapy (see, naptumomab estafenatox, discussed below). Superantigens expressed by these polynucleotide sequences may be wild-type superantigens, modified superantigens, or wild-type or modified superantigens conjugated or fused with targeting moieties. The superantigens may be administered to a mammal, such as a human, directly, for example by injection, or may be delivered, for example, by exposure of blood of a patient to the superantigen outside the body, or, for example, via placing a gene encoding a superantigen inside a mammal to be treated (e.g., via known gene therapy methods and vectors such as, for example, via cells containing, and capable of expressing, the gene) and expressing the gene within the mammal.

Examples of superantigens and their administration to mammals are described in the following U.S. patents and patent applications: U.S. Pat. Nos. 5,858,363, 6,197,299, 6,514,498, 6,713,284, 6,692,746, 6,632,640, 6,632,441, 6,447,777, 6,399,332, 6,340,461, 6,338,845, 6,251,385, 6,221,351, 6,180,097, 6,126,945, 6,042,837, 6,713,284, 6,632,640, 6,632,441, 5,859,207, 5,728,388, 5,545,716, 5,519,114, 6,926,694, 7,125,554, 7,226,595, 7,226,601, 7,094,603, 7,087,235, 6,835,818, 7,198,398, 6,774,218, 6,913,755, 6,969,616, and 6,713,284, U.S. Patent Application Nos. 2003/0157113, 2003/0124142, 2002/0177551, 2002/0141981, 2002/0115190, and 2002/0051765, and International (PCT) Publication No. WO/03/094846.

B. Modified Superantigens

Within the scope of this invention, superantigens may be engineered in a variety of ways, including modifications that retain or enhance the ability of a superantigen to stimulate T lymphocytes, and may, for example, alter other aspects of the superantigen, such as, for example, its seroreactivity or immunogenicity. Modified superantigens include synthetic molecules that have superantigen activity (i.e., the ability to activate subsets of T lymphocytes).

It is contemplated that various changes may be made to the polynucleotide sequences encoding a superantigen without appreciable loss of its biological utility or activity, namely the induction of the T-cell response to result in cytotoxicity of the tumor cells. Furthermore, the affinity of the superantigen for the MHC class II molecule can be decreased with minimal effects on the cytotoxicity of the superantigen. This, for example, can help to reduce toxicity that may otherwise occur if a superantigen retains its wild-type ability to bind MHC class II antigens (as in such a case, class II expressing cells, such as immune system cells, could also be affected by the response to the superantigen).

Techniques for modifying superantigens (e.g., polynucleotides and polypeptides), including for making synthetic superantigens, are well known in the art and include, for example PCR mutagenesis, alanine scanning mutagenesis, and site-specific mutagenesis (see, U.S. Pat. Nos. 5,220,007; 5,284,760; 5,354,670; 5,366,878; 5,389,514; 5,635,377; and 5,789,166).

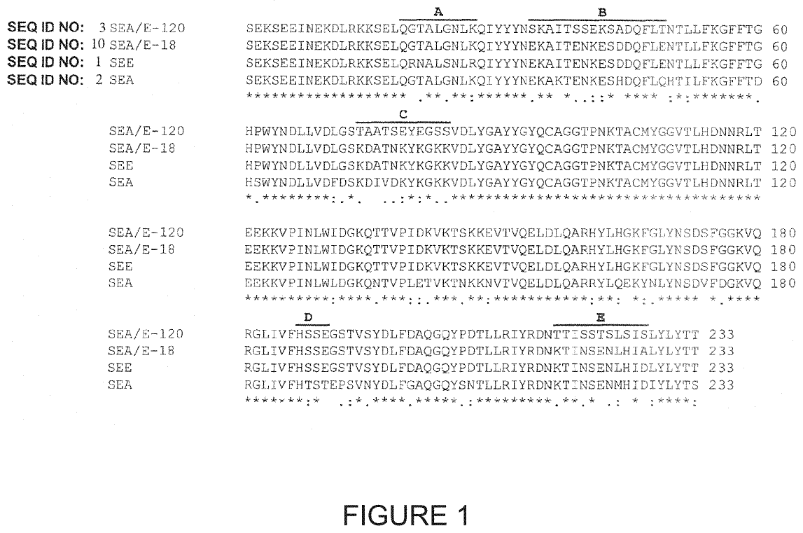

In some embodiments, a superantigen may be modified such that its seroreactivity is reduced compared to a reference wild-type superantigen, but its ability to activate T-cells is retained or enhanced relative to wild-type. One technique for making such modified superantigens includes substituting certain amino acids in certain regions from one superantigen to another. This is possible because many superantigens, including but not limited to, SEA, SEE, and SED, share sequence homology in certain areas that have been linked to certain functions (Marrack and Kappler (1990) SCIENCE 248(4959): 1066; see also FIG. 1, which shows region of homology between different wild type and engineered superantigens). For example, in certain embodiments of the present invention, a superantigen that has a desired T-cell activation-inducing response, but a non-desired high seroreactivity, is modified such that the resulting superantigen retains its T-cell activation ability but has reduced seroreactivity.

It is known and understood by those of skill in the art that the sera of humans normally contain various titers of antibodies against superantigens. For the staphylococcal superantigens, for instance, the relative titers are TSST-1>SEB>SEC-1>SE3>SEC2>SEA>SED>SEE. As a result, the seroreactivity of, for example, SEE (Staphylococcal enterotoxin E) is lower than that of, for example, SEA (Staphylococcal enterotoxin A). Based on this data, one skilled in the art may prefer to administer a low titer superantigen, such as, for example SEE, instead of a high titer superantigen, such as, for example, SEB (Staphylococcal enterotoxin B). However, as has also been discovered, different superantigens have differing T-cell activation properties relative to one another, and for wild-type superantigens, the best T-cell activating superantigens often also have undesirably high seroreactivity.

These relative titers sometimes correspond to potential problems with seroreactivity, such as problems with neutralizing antibodies. Thus, the use of a low titer superantigen, such as SEA or SEE may be helpful in reducing or avoiding seroreactivity of parenterally administered superantigens. A low titer superantigen has a low seroreactivity as measured, for example, by typical anti-superantigen antibodies in a general population. In some instances it may also have a low immunogenicity. Such low titer superantigens may be modified to retain its low titer as described herein.

Approaches for modifying superantigens can be used to create superantigens that have both the desired T-cell activation properties and reduced seroreactivity, and in some instances also reduced immunogenicity. Given that certain regions of homology between superantigens relate to seroreactivity, it is possible to engineer a recombinant superantigen that has a desired T-cell activation and a desired seroreactivity and/or immunogenicity. Furthermore, the protein sequences and immunological cross-reactivity of the superantigens or staphylococcal enterotoxins are divided into two related groups. One group consists of SEA, SEE and SED. The second group is SPEA, SEC and SEB. Thus, it is possible to select low titer superantigens to decrease or eliminate the cross-reactivity with high titer or endogenous antibodies directed against staphylococcal enterotoxins.

Regions in the superantigens that are believed to play a role in seroreactivity include, for example, Region A, which comprises amino acid residues 20, 21, 22, 23, 24, 25, 26, and 27; Region B, which comprises amino acid residues 34, 35, 36, 37, 38, 39, 40, 41, 42, 43, 44, 45, 46, 47, 48, and 49; Region C, which comprises amino acid residues 74, 75, 76, 77, 78, 79, 80, 81, 82, 83, and 84; Region D, which comprises amino acid residues 187, 188, 189 and 190; and Region E, which comprise the amino acid residues, 217, 218, 219, 220, 221, 222, 223, 224, 225, 226, and 227 (see, U.S. Pat. No. 7,125,554, and FIG. 1 herein). Thus, it is contemplated that these regions can be mutated using, for example amino acid substitution, to produce a superantigen having altered seroreactivity.

Polypeptide or amino acid sequences for the above listed superantigens can be obtained from any sequence data bank, for example Protein Data Bank and/or GenBank. Exemplary GenBank accession numbers include, but are not limited to, SEE is P12993; SEA is P013163; SEB is P01552; SEC1 is P01553; SED is P20723; and SEH is AAA19777.

In certain embodiments of the present invention, the wild-type SEE sequence (SEQ ID NO: 1) or the wild type SEA sequence (SEQ ID NO: 2) can be modified such that amino acids in any of the identified regions A-E (see, FIG. 1) are substituted with other amino acids. Such substitutions include for example, K79, K81, K83 and D227 or K79, K81, K83, K84 and D227, or, for example, K79E, K81E, K83S and D227S or K79E, K81E, K83S, K84S and D227A. In certain embodiments, the superantigen is SEA/E-120 (SEQ ID NO: 3; see also U.S. Pat. No. 7,125,554) or SEAD227A (SEQ ID NO: 4; see also U.S. Pat. No. 7,226,601).

1. Modified Polynucleotides and Polypeptides

A biological functional equivalent of a polynucleotide encoding a naturally occurring or a reference superantigen may comprise a polynucleotide that has been engineered to contain distinct sequences while at the same time retaining the capacity to encode the naturally occurring or reference superantigen. This can be accomplished due to the degeneracy of the genetic code, i.e., the presence of multiple codons, which encode for the same amino acids. In one example, it is possible to introduce a restriction enzyme recognition sequence into a polynucleotide while not disturbing the ability of that polynucleotide to encode a protein. Other polynucleotide sequences may encode superantigens that are different but functionally substantially equivalent in at least one biological property or activity (for example, at least 50%, 60%, 70%, 80%, 90%, 95%, 98% of the biological property or activity, for example, without limitation, the ability to induce a T-cell response that results in cytotoxicity of the tumor cells) to a reference superantigen.

In another example, a polynucleotide may be (and encode) a superantigen functionally equivalent to a reference superantigen even though it may contain more significant changes. Certain amino acids may be substituted for other amino acids in a protein structure without appreciable loss of interactive binding capacity with structures such as, for example, antigen-binding regions of antibodies, binding sites on substrate molecules, receptors, and such like. Furthermore, conservative amino acid replacements may not disrupt the biological activity of the protein, as the resultant structural change often is not one that impacts the ability of the protein to carry out its designed function. It is thus contemplated that various changes may be made in the sequence of genes and proteins disclosed herein, while still fulfilling the goals of the present invention.

Amino acid substitutions may be designed to take advantage of the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents. In addition, it may be possible to introduce non-naturally occurring amino acids. Approaches for making amino acid substitutions with other naturally occurring and non-naturally occurring amino acid are described in U.S. Pat. No. 7,763,253.

In terms of functional equivalents, it is understood that, implicit in the definition of a “biologically functional equivalent” protein and/or polynucleotide, is the concept that there is a limited number of changes that may be made within a defined portion of the molecule while retaining a molecule with an acceptable level of equivalent biological activity. Biologically functional equivalents are thus considered to be those proteins (and polynucleotides) where selected amino acids (or codons) may be substituted without substantially affecting biological function. Functional activity includes the induction of the T-cell response to result in cytotoxicity of the tumor cells.

In addition, it is contemplated that a modified superantigen can be created by substituting homologous regions of various proteins via “domain swapping,” which involves the generation of chimeric molecules using different but, in this case, related polypeptides. By comparing various superantigen proteins to identify functionally related regions of these molecules (see, e.g., FIG. 1), it is possible to swap related domains of these molecules so as to determine the criticality of these regions to superantigen function. These molecules may have additional value in that these “chimeras” can be distinguished from natural molecules, while possibly providing the same function.

In certain embodiments, the superantigen comprises an amino acid sequence that is at least 70%, 75%, 80%, 85%, 90%, 95%, 98%, or 99% identical to the sequence of a reference superantigen selected from SEQ ID NO: 1, SEQ ID NO: 2, SEQ ID NO: 3, and SEQ ID NO: 4, wherein the superantigen optionally retains at least 50%, 60%, 70% 80%, 90%, 95%. 98%, 99%, or 100% of a biological activity or property of the reference superantigen.

In certain embodiments, the superantigen comprises an amino acid sequence that is encoded by a nucleic acid that is at least 70%, 75%, 80%, 85%, 90%, 95%, 98%, or 99% identical to a nucleic acid encoding the superantigen selected from SEQ ID NO: 1, SEQ ID NO: 2, SEQ ID NO: 3, and SEQ ID NO: 4, wherein the superantigen optionally retains at least 50%, 60%, 70% 80%, 90%, 95%. 98%, 99%, or 100% of a biological activity or property of the reference superantigen.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; −W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=15 for all programs, not applicable to blastn; and −Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

C. Targeted Superantigens

In order to increase specificity, the superantigen preferably is conjugated to a targeting moiety to create a targeted superantigen conjugate that binds an antigen preferentially expressed by a cancer cell, for example, a cell surface antigen such as 5T4. The targeting moiety is a vehicle that can be used to bind superantigen to the cancerous cells, for example, the surface of the cancerous cells. The targeted superantigen conjugate should retain the ability to activate large numbers of T lymphocytes. For example, the targeted superantigen conjugate should activate large numbers of T-cells and direct them to tissues containing the tumor-associated antigen bound to the targeting moiety. In such situations, specific target cells are preferentially killed, leaving the rest of the body relatively unharmed. This type of therapy is desirable, as non-specific anti-cancer agents, such as cytostatic chemotherapeutic drugs, are nonspecific and kill large numbers of cells not associated with tumors to be treated. For example, studies with targeted superantigen conjugates have shown that inflammation with infiltration by cytotoxic T lymphocytes (CTLs) into tumor tissue increases rapidly in response to the first injection of a targeted superantigen (Dohlsten et al. (1995) PROC. NATL. ACAD. SCI. USA 92:9791-9795). This inflammation with infiltration of CTLs into the tumor is one of the major effectors of the anti-tumor therapeutic of targeted superantigens.

Tumor-targeted superantigens represent an immunotherapy against cancer and are therapeutic fusion proteins containing a targeting moiety conjugated to a superantigen (Dohlsten et al. (1991) PROC. NATL. ACAD. SCI. USA 88:9287-9291; Dohlsten et al. (1994) PROC. NATL. ACAD. SCI. USA 91:8945-8949).

The targeting moiety can in principle be any structure that is able to bind to a cellular molecule, for example, a cell surface molecule and preferably is a disease specific molecule. The targeted molecule (e.g., antigen) against which the targeting moiety is directed is usually different from (a) the VP chain epitope to which superantigen binds, and (b) the MHC class II epitopes to which superantigens bind. The targeting moiety can be selected from antibodies, including antigen binding fragments thereof, soluble T-cell receptors, growth factors, interleukins (e.g., interleukin-2), hormones, etc.

In certain preferred embodiments, the targeting moiety is an antibody (e.g., Fab, F(ab)₂, Fv, single chain antibody, etc.). Antibodies are extremely versatile and useful cell-specific targeting moieties because they typically can be generated against any cell surface antigen of interest. Monoclonal antibodies have been generated against cell surface receptors, tumor-associated antigens, and leukocyte lineage-specific markers such as CD antigens. Antibody variable region genes can be readily isolated from hybridoma cells by methods well known in the art. Exemplary tumor-associated antigens that can be used to produce a targeting moiety can include, but are not limited to gp100, Melan-A/MART, MAGE-A, MAGE (melanoma antigen E), MAGE-3, MAGE-4, MAGEA3, tyrosinase, TRP2, NY-ESO-1, CEA (carcinoembryonic antigen), PSA, p53, Mammaglobin-A, Survivin, MUC1 (mucin1)/DF3, metallopanstimulin-1 (MPS-1), Cytochrome P450 isoform 1B1, 90K/Mac-2 binding protein, Ep-CAM (MK-1), HSP-70, hTERT (TRT), LEA, LAGE-1/CAMEL, TAGE-1, GAGE, 5T4, gp70, SCP-1, c-myc, cyclin B1, MDM2, p62, Koc, IMP1, RCAS1, TA90, OA1, CT-7, HOM-MEL-40/SSX-2, SSX-1, SSX-4, HOM-TES-14/SCP-1, HOM-TES-85, HDAC5, MBD2, TRIP4, NY--CO-45, KNSL6, HIP1R, Seb4D, KIAA1416, IMP1, 90K/Mac-2 binding protein, MDM2, NY/ESO, EGFRvIII, IL-13Rα2, HER2, GD2, EGFR, PDL1, Mesothelin, PSMA, TGFβRDN, LMP1, GPC3, Fra, MG7, CD133, CMET, PSCA, Glypican3, ROR1, NKR-2, CD70 and LMNA.

Exemplary cancer-targeting antibodies can include, but are not limited to, anti-CD19 antibodies, anti-CD20 antibodies, anti-5T4 antibodies, anti-Ep-CAM antibodies, anti-Her-2/neu antibodies, anti-EGFR antibodies, anti-CEA antibodies, anti-prostate specific membrane antigen (PSMA) antibodies, and anti-IGF-1R antibodies. It is understood that the superantigen can be conjugated to an immunologically reactive antibody fragment such as C215Fab, 5T4Fab (see, WO8907947) or C242Fab (see, WO9301303).

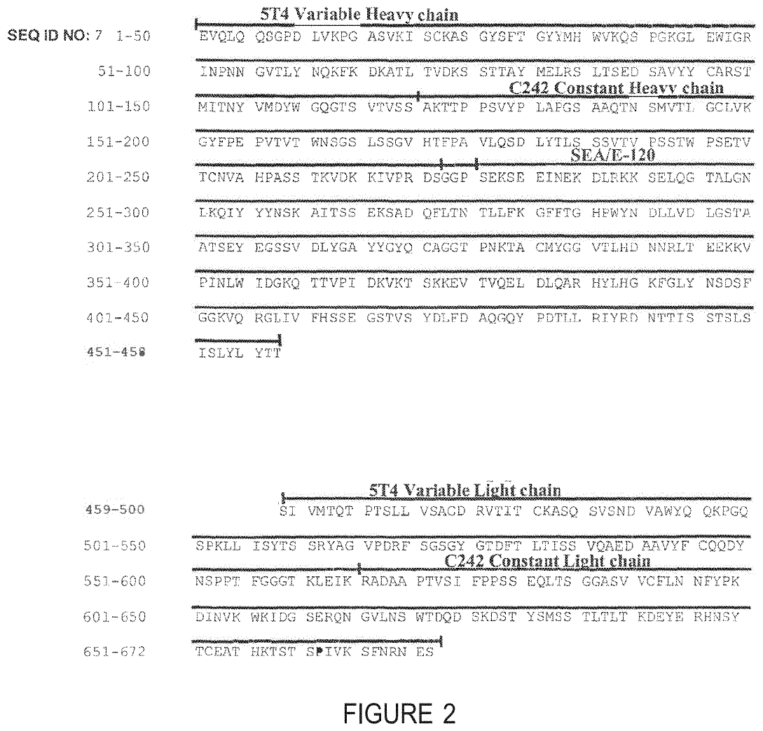

Examples of tumor targeted superantigens that can be used in the present invention include C215Fab-SEA (SEQ ID NO: 5), 5T4Fab-SEA_{D227A} (SEQ ID NO: 6) and 5T4Fab-SEA/E-120 (SEQ ID NO: 7, see FIG. 1 and FIG. 2).

In a preferred embodiment, a preferred conjugate is a superantigen conjugate known as naptumomab estafenatox, which is the fusion protein of the Fab fragment of an anti-5T4 antibody and the SEA/E-120 superantigen. Naptumomab estafenatox comprises two protein chains that cumulatively include an engineered Staphylococcal enterotoxin superantigen (SEA/E-120) and a targeting 5T4 Fab comprising modified 5T4 variable region sequences fused to the constant region sequences of the murine IgG1/κ antibody C242. The first protein chain comprises residues 1 to 458 of SEQ ID NO: 7 (see also, SEQ ID NO: 8), and includes a chimeric 5T4 Fab heavy chain, corresponding to residues 1 to 222 of SEQ ID NO: 7, and the SEA/E-120 superantigen, corresponding to residues 226 to 458 of SEQ ID NO: 7, covalently linked via a GGP tripeptide linker, corresponding to residues 223-225 of SEQ ID NO: 7. The second chain comprises residues 459 to 672 of SEQ ID NO: 7 (see also, SEQ ID NO: 9) and includes a chimeric 5T4 Fab light chain. The two protein chains are held together by non-covalent interactions between the Fab heavy and light chains. Residues 1-458 of SEQ ID NO: 7 correspond to residues 1-458 of SEQ ID NO: 8, and residues 459-672 of SEQ ID NO: 7 correspond to residues 1-214 of SEQ ID NO: 9. Naptumomab estafenatox comprises the proteins of SEQ ID NOS: 8 and 9 held together by non-covalent interactions between the Fab heavy and Fab light chains. Naptumomab estafenatox induces T-cell mediated killing of cancer cells at concentrations around 10 pM and the superantigen component of the conjugate has been engineered to have low binding to human antibodies and MHC Class II.

It is contemplated that other antibody based targeting moieties can be designed, modified, expressed, and purified using techniques known in the art and discussed in more detail below.

Another type of targeting moiety includes a soluble T-cell receptor (TCR). Some forms of soluble TCR may contain either only extracellular domains or extracellular and cytoplasmic domains. Other modifications of the TCR may also be envisioned to produce a soluble TCR in which the transmembrane domains have been deleted and/or altered such that the TCR is not membrane bound as described in U.S. Publication Application Nos. U.S. 2002/119149, U.S. 2002/0142389, U.S. 2003/0144474, and U.S. 2003/0175212, and International Publication Nos. WO2003020763; WO9960120 and WO9960119.

The targeting moiety can be conjugated to the superantigen by using either recombinant techniques or chemically linking of the targeting moiety to the superantigen.

1. Recombinant Linker (Fusion Protein)

It is contemplated that a gene encoding a superantigen linked directly or indirectly (for example, via an amino acid containing linker) to a targeting moiety can be created and expressed using conventional recombinant DNA technologies. For example, the amino terminal of a modified superantigen can be linked to the carboxy terminal of a targeting moiety or vice versa. For antibodies, or antibody fragments that may serve as targeting moieties, either the light or the heavy chain may be utilized for creating a fusion protein. For example, for a Fab fragment, the amino terminus of the modified superantigen can be linked to the first constant domain of the heavy antibody chain (CH₁). In some instances, the modified superantigen can be linked to a Fab fragment by linking the VH and VL domain to the superantigen. Alternatively, a peptide linker can be used to join the superantigen and targeting moiety together. When a linker is employed, the linker preferably contains hydrophilic amino acid residues, such as Gln, Ser, Gly, Glu, Pro, His and Arg. Preferred linkers are peptide bridges consisting of 1-10 amino acid residues, more particularly, 3-7 amino acid residues. An exemplary linker is the tripeptide-GlyGlyPro-. These approaches have been used successfully in the design and manufacture of the naptumomab estafenatox superantigen conjugate.

2. Chemical Linkage

It is also contemplated that the superantigen may be linked to the targeting moiety via a chemical linkage. Chemical linkage of the superantigen to the targeting moiety may require a linker, for example, a peptide linker. The peptide linker preferably is hydrophilic and exhibits one or more reactive moieties selected from amides, thioethers, disulfides etc. (See, e.g., U.S. Pat. Nos. 5,858,363, 6,197,299, and 6,514,498). It is also contemplated that the chemical linkage can use homo- or heterobifunctional crosslinking reagents. Chemical linking of a superantigen to a targeting moiety often utilizes functional groups (e.g., primary amino groups or carboxy groups) that are present in many positions in the compounds.

D. Expression of Superantigens and Superantigen Conjugates

When recombinant DNA technologies are employed, the superantigen or the superantigen-targeting moiety conjugate may be expressed using standard expression vectors and expression systems. The expression vectors, which have been genetically engineered to contain the nucleic acid sequence encoding the superantigen, are introduced (e.g., transfected) into host cells to produce the superantigen (see, e.g. Dohlsten et al. (1994), Forsberg et al. (1997) J. BIOL. CHEM. 272:12430-12436, Erlandsson et al. (2003) J. MOL. BIOL. 333:893-905 and WO2003002143).

Host cells can be genetically engineered, for example, by transformation or transfection technologies, to incorporate nucleic acid sequences and express the superantigen. Introduction of nucleic acid sequences into the host cell can be affected by calcium phosphate transfection, DEAE-dextran mediated transfection, microinjection, cationic lipid-mediated transfection, electroporation, transduction, scrape loading, ballistic introduction, infection or other methods. Such methods are described in many standard laboratory manuals, such as, Davis et al. (1986) BASIC METHODS IN MOLECULAR BIOLOGY and Sambrook, et al. (1989) MOLECULAR CLONING: A LABORATORY MANUAL, 2nd Ed., Cold Spring Harbor Laboratory Press, Cold Spring Harbor, N.Y.

Representative examples of appropriate host cells include bacterial cells, such as streptococci, staphylococci, E. coli, Streptomyces and Bacillus subtilis cells; fungal cells, such as yeast cells and aspergillus cells; insect cells such as Drosophila S2 and Spodoptera Sf9 cells; mammalian cells such as CHO, COS, HeLa, C127, 3T3, BHK, HEK-293 and Bowes melanoma cells.

Examples of production systems for superantigens are found, for example, in U.S. Pat. No. 6,962,694.

E. Protein Purification

The superantigen and/or the superantigen-targeting moiety conjugates preferably are purified prior to use, which can be accomplished using a variety of purification protocols. Having separated the superantigen or the superantigen-targeting moiety conjugate from other proteins, the protein of interest may be further purified using chromatographic and electrophoretic techniques to achieve partial or complete purification (or purification to homogeneity). Analytical methods particularly suited to the preparation of a pure peptide are ion-exchange chromatography, size exclusion chromatography; affinity chromatography; polyacrylamide gel electrophoresis; isoelectric focusing. The term “purified” as used herein, is intended to refer to a composition, isolatable from other components, wherein the macromolecule (e.g., protein) of interest is purified to any degree relative to its original state. Generally, the terms “purified” refer to a macromolecule that has been subjected to fractionation to remove various other components, and which substantially retains its biological activity. The term “substantially purified” refers to a composition in which the macromolecule of interest forms the major component of the composition, such as constituting about 60%, about 70%, about 80%, about 90%, about 95% or more of the content of the composition.

Various methods for quantifying the degree of purification of the protein are known to those of skill in the art, including, for example, determining the specific activity of an active fraction, and assessing the amount of a given protein within a fraction by SDS-PAGE analysis, High Performance Liquid Chromatography (HPLC), or any other fractionation technique. Various techniques suitable for use in protein purification include, for example, precipitation with ammonium sulfate, PEG, antibodies and the like or by heat denaturation, followed by centrifugation; chromatography steps such as ion exchange, gel filtration, reverse phase, hydroxyapatite, affinity chromatography; isoelectric focusing; gel electrophoresis; and combinations of such and other techniques. It is contemplated that the order of conducting the various purification steps may be changed, or that certain steps may be omitted, and still result in a suitable method for the preparation of a substantially purified protein or peptide.

### III. B-Cell Depleting Agent

It is contemplated that, in certain embodiments, the efficacy of the superantigen conjugate can be enhanced by administering the superantigen conjugate to the subject to be treated together with a B-cell depleting agent.

As used herein, a “B-cell depleting agent” refers to any agent that reduces the number of B-cells, e.g., peripheral B-cells, in a subject (or in a cell, fluid, or tissue sample from the subject). For example, administration of a B-cell depleting agent to a subject may reduce the amount of B-cells, e.g., peripheral B-cells, in the subject (or in the cell, fluid, or tissue sample from the subject) by at least about 10%, at least about 20%, at least about 30%, at least about 40%, at least about 50%, at least about 60%, at least about 70%, at least about 80%, at least about 90%, or at least about 95% relative to the amount of B-cells, e.g., peripheral B-cells, in the subject (or in the cell, fluid, or tissue sample from the subject) prior to administration of the B-cell depleting agent.

The number of B-cells in a subject may be determined by any method known in the art, including, for example, flow cytometric, immunohistochemical or immunofluorescent methods, using antibodies against B-cell markers such as CD20, CD19, PAX5, and/or B220/CD45R. The number of B-cells in a subject may also be determined by quantification of protein or mRNA levels of B-cell markers in the subject or in a cell, fluid, or tissue sample from the subject. Exemplary methods for the quantification of protein levels include enzyme-linked immunosorbent assay (ELISA) or Western Blot, and exemplary methods for quantification of mRNA levels include quantitative RT-PCR or microarray technologies. In certain embodiments, the number of B-cells in a subject may be determined by staining for B220/CD45R as described in Example 1 herein.

In certain embodiments, the B-cell depleting agent is an anti-CD20 antibody. CD20 (also known as B-lymphocyte antigen CD20, B-lymphocyte surface antigen B1, human B-lymphocyte-restricted differentiation antigen, Leu-16, Bp35, BMS, and LF5) is a hydrophobic transmembrane protein with a molecular weight of approximately 35 kD expressed on pre-B and mature B lymphocytes. CD20 plays a role in the development and differentiation of B-cells into plasma cells. Exemplary anti-CD20 antibodies include ibritumomab, obinutuzumab, ocaratuzumab, ocrelizumab, ofatumumab, rituximab, veltuzumab, tositumomab, ublituximab, veltuzumab, PRO131921, and TRU-015.

In certain embodiments, the anti-CD20 antibody is a Type I anti-CD20 antibody (as described in Cragg et al. (2004) BLOOD 103:2738-2743, Cragg et al. (2003) BLOOD 101: 1045-1052, Klein et al. (2013) MABS 5:22-33, and U.S. Patent Application Publication No. US20170209573A1). Type I anti-CD20 antibodies bind to a class I CD20 epitope, primarily utilize complement to kill target cells, localize CD20 to lipid rafts, show high CDC activity, show full binding capacity to B-cells, and/or show only weak induction of homotypic aggregation and direct cell death. Examples of type I anti-CD20 antibodies include rituximab, ofatumumab, veltuzumab, ocaratuzumab, ocrelizumab, PRO131921, ublituximab, HI47 IgG3 (ECACC, hybridoma), 2C6 IgG1 (as disclosed in International (PCT) Publication No. WO2005/103081), 2F2 IgG1 (as disclosed in International (PCT) Publication Nos. WO2004/035607 and WO2005/103081) and 2H7 IgG1 (as disclosed in International (PCT) Publication No. WO2004/056312).

In certain embodiments, the anti-CD20 antibody is a Type II anti-CD20 antibody (as described in Cragg et al. (2004) BLOOD 103:2738-2743, Cragg et al. (2003) BLOOD 101: 1045-1052, Klein et al. (2013) MABS 5:22-33, and U.S. Patent Application Publication No. US20170209573A1). Type II anti-CD20 antibodies bind to a class II CD20 epitope, primarily operate through direct induction of cell death, do not localize CD20 to lipid rafts, show low CDC activity, show only about half the binding capacity to B-cells as compared to Type I anti-CD20 antibodies, and/or induce homotypic aggregation and direct cell death. Examples of type II anti-CD20 antibodies include obinutuzumab (GA101), tositumumab (B1), humanized B-Ly1 antibody IgG1 (a chimeric humanized IgG1 antibody as disclosed in International (PCT) Publication Nos. WO2005/044859 and WO2007/031875), 11B8 IgG1 (as disclosed in International (PCT) Publication No. WO2004/035607) and AT80 IgG1.

In certain embodiments, the anti-CD20 antibody is an IgG antibody, e.g., an IgG1 antibody. In certain embodiments, the anti-CD20 antibody is engineered to have an increased proportion of non-fucosylated oligosaccharides in the Fc region as compared to a non-engineered antibody. In certain embodiments, at least about 40% of the N-linked oligosaccharides in the Fc region of the anti-CD20 antibody are non-fucosylated.

In certain embodiments, the anti-CD20 antibody comprises: (i) an immunoglobulin heavy chain variable region comprising a CDRH1 comprising the amino acid sequence of SEQ ID NO: 11, a CDRH2 comprising the amino acid sequence of SEQ ID NO: 12, and a CDRH3 comprising the amino acid sequence of SEQ ID NO: 13; and (ii) an immunoglobulin light chain variable region comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16. In certain embodiments, the CDRs are interposed between human or humanized immunoglobulin framework regions. In certain embodiments, the anti-CD20 antibody competes for binding to CD20, or binds to the same epitope on CD20, as an antibody comprising (i) an immunoglobulin heavy chain variable region comprising a CDRH1 comprising the amino acid sequence of SEQ ID NO: 11, a CDRH2 comprising the amino acid sequence of SEQ ID NO: 12, and a CDRH3 comprising the amino acid sequence of SEQ ID NO: 13; and (ii) an immunoglobulin light chain variable region comprising a CDRL1 comprising the amino acid sequence of SEQ ID NO: 14, a CDRL2 comprising the amino acid sequence of SEQ ID NO: 15, and a CDRL3 comprising the amino acid sequence of SEQ ID NO: 16.

In certain embodiments, the anti-CD20 antibody comprises: (i) an immunoglobulin heavy chain variable region comprising the amino acid sequence of SEQ ID NO: 17, or an amino acid sequence that has at least 85%, 90%, 95%, 96%, 97%, 98%, or 99% sequence identity to SEQ ID NO: 17, and (ii) an immunoglobulin light chain variable region comprising the amino acid sequence of SEQ ID NO: 18, or an amino acid sequence that has at least 85%, 90%, 95%, 96%, 97%, 98%, or 99% sequence identity to SEQ ID NO: 18. In certain embodiments, the anti-CD20 antibody further comprises a heavy chain constant region. In certain embodiments, the anti-CD20 antibody competes for binding to CD20, or binds to the same epitope on CD20, as an antibody comprising: (i) an immunoglobulin heavy chain variable region comprising the amino acid sequence of SEQ ID NO: 17, or an amino acid sequence that has at least 85%, 90%, 95%, 96%, 97%, 98%, or 99% sequence identity to SEQ ID NO: 17, and (ii) an immunoglobulin light chain variable region comprising the amino acid sequence of SEQ ID NO: 18, or an amino acid sequence that has at least 85%, 90%, 95%, 96%, 97%, 98%, or 99% sequence identity to SEQ ID NO: 18.

In certain embodiments, the anti-CD20 antibody comprises: (i) an immunoglobulin heavy chain comprising the amino acid sequence of SEQ ID NO: 19, or an amino acid sequence that has at least 85%, 90%, 95%, 96%, 97%, 98%, or 99% sequence identity to SEQ ID NO: 19, and (ii) an immunoglobulin light chain comprising the amino acid sequence of SEQ ID NO: 20, or an amino acid sequence that has at least 85%, 90%, 95%, 96%, 97%, 98%, or 99% sequence identity to SEQ ID NO: 20. In certain embodiments, the anti-CD20 antibody competes for binding to CD20, or binds to the same epitope on CD20, as an antibody comprising: (i) an immunoglobulin heavy chain comprising the amino acid sequence of SEQ ID NO: 19, or an amino acid sequence that has at least 85%, 90%, 95%, 96%, 97%, 98%, or 99% sequence identity to SEQ ID NO: 19, and (ii) an immunoglobulin light chain comprising the amino acid sequence of SEQ ID NO: 20, or an amino acid sequence that has at least 85%, 90%, 95%, 96%, 97%, 98%, or 99% sequence identity to SEQ ID NO: 20.

In certain embodiments, the anti-CD20 antibody is obinutuzumab (also known as GAZYVA®, GAZYVARO®, or GA101). In certain embodiments, the anti-CD20 antibody is tositumomab. Additional exemplary anti-CD20 antibodies, and methods of use, are described in U.S. Patent Application Publication No. US20170209573A1.

Additional exemplary B-cell depleting agents include anti-CD19 antibodies (e.g., blinatumomab, inebilizumab, tafasitamab), anti-CD21 antibodies (e.g., OKB-7), anti-CD22 antibodies (e.g., epratuzumab, inotuzumab, moxetumomab), BLyS inhibitors (e.g., atacicept, belimumab, blisibimod, BR3-Fc), and chemotherapeutic agents (e.g., gemcitabine).

### IV. Immunopotentiator

It is contemplated that, in certain embodiments, the efficacy of the superantigen conjugate and/or B-cell depleting agent can be enhanced by further administering an immunopotentiator to the subject to be treated.

In certain embodiments, exemplary immunopotentiators can: (a) stimulate activating T-cell signaling, (b) repress T-cell inhibitory signalling between the cancerous cells and a T-cell, (c) repress inhibitory signalling that leads to T-cell expansion, activation and/or activity via a non-human IgG1-mediated immune response pathway, for example, a human IgG4 immunoglobulin-mediated pathway, (d) a combination of (a) and (b), (e) combination of (a) and (c), (f) a combination of (b) and (c), and (g) a combination of (a), (b), and (c).

In certain embodiments, the immunopotentiator is a checkpoint pathway inhibitor. A number of T-cell checkpoint inhibitor pathways have been identified to date, for example, the PD-1 immune checkpoint pathway and Cytotoxic T-lymphocyte antigen-4 (CTLA-4) immune checkpoint pathway.

PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, PD-L2, etc., that interact with PD-1 on the surface of T-cells to shut down or modulate T-cell activity. Using this approach, cancer can evade the T-cell mediated immune response.

In the CTLA-4 pathway, the interaction of CTLA-4 on the T-cell with its ligands (e.g., CD80, also known as B7-1, and CD86) on the surface of an antigen presenting cells (rather than the cancer calls) leads to T-cell inhibition. As a result, the ligand that inhibits T-cell activation or activity (e.g., CD80 or CD86) is provided by an antigen presenting cell (a key cell type in the immune system) rather than the cancer cell. Although CTLA-4 and PD-1 binding both have similar negative effects on T-cells the timing of downregulation, the responsible signaling mechanisms, and the anatomic locations of immune inhibition by these two immune checkpoints differ (American Journal of Clinical Oncology. Volume 39, Number 1, February 2016). Unlike CTLA-4, which is confined to the early priming phase of T-cell activation, PD-1 functions much later during the effector phase, (Keir et al. (2008) ANNU. REV IMMUNOL., 26:677-704). CTLA-4 and PD-1 represent two T-cell-inhibitory receptors with independent, non-redundant mechanisms of action.

In certain embodiments, the immunopotentiator prevents (completely or partially) an antigen expressed by the cancerous cell from repressing T-cell inhibitory signaling between the cancerous cell and the T-cell. In one embodiment, such an immunopotentiator is a checkpoint inhibitor, for example, a PD-1-based inhibitor. Examples of such immunopotentiators include, for example, anti-PD-1 antibodies, anti-PD-L1 antibodies, and anti-PD-L2 antibodies.

In certain embodiments, the superantigen conjugate is administered with a PD-1-based inhibitor. A PD-1-based inhibitor can include (i) a PD-1 inhibitor, i.e., a molecule (for example, an antibody or small molecule) that binds to PD-1 on a T-cell to prevent the binding of a PD-1 ligand expressed by the cancer cell of interest, and/or (ii) a PD-L inhibitor, e.g., a PD-L1 or PD-L2 inhibitor, i.e., a molecule (for example, an antibody or small molecule) that binds to a PD-1 ligand (for example, PD-L1 or PD-L2) to prevent the PD-1 ligand from binding to its cognate PD-1 on the T-cell.

In certain embodiments the superantigen conjugate is administered with a CTLA-4 inhibitor, e.g., an anti-CTLA-4 antibody. Exemplary anti-CTLA-4 antibodies include ipilimumab and tremelimumab.

In certain embodiments, the immunopotentiator prevents (completely or partially) an antigen expressed by the cancerous cell from repressing T-cell expansion, activation and/or activity via a human IgG4 (a non-human IgG1) mediated immune response pathway, for example, not via an ADCC pathway. It is contemplated that, in such embodiments, although the immune response potentiated by the superantigen conjugate and the immunopotentiator may include some ADCC activity, the principal component(s) of the immune response do not involve ADCC activity. In contrast, some of the antibodies currently being used in immunotherapy, such as ipilimumab (an anti-CTLA-4 IgG1 monoclonal antibody), can kill targeted cells via ADCC through signaling via their Fc domain through Fc receptors on effector cells. Ipilimumab, like many other therapeutic antibodies, was designed as a human IgG1 immunoglobulin, and although ipilimumab blocks interactions between CTLA-4 and CD80 or CD86, its mechanism of action is believed to include ADCC depletion of tumor-infiltrating regulatory T-cells that express high levels of cell surface CTLA-4. (Mahoney et al. (2015) NATURE REVIEWS, DRUG DISCOVERY 14: 561-584.) Given that CTLA-4 is highly expressed on a subset of T-cells (regulatory T-cells) that act to negatively control T-cells activation, when an anti-CTLA-4 IgG1 antibody is administered, the number of regulatory T-cells is reduced via ADCC.

In certain embodiments, it is desirable to use immunopotentiators whose mode of action is primarily to block the inhibitory signals between the cancer cells and the T-cells without significantly depleting the T-cell populations (for example, permitting the T-cell populations to expand). To achieve this, it is desirable to use an antibody, for example, an anti-PD-1 antibody, an anti-PD-L1 antibody or an anti-PD-L2 antibody, that has or is based on a human IgG4 isotype. Human IgG4 isotype is preferred under certain circumstances because this antibody isotype invokes little or no ADCC activity compared to the human IgG1 isotype (Mahoney et al. (2015) supra). Accordingly, in certain embodiments, the immunopotentiator, e.g., the anti-PD-1 antibody, anti-PD-L1 antibody, or anti-PD-L2 antibody has or is based on a human IgG4 isotype. In certain embodiments, the immunopotentiator is an antibody not known to deplete Tregs, e.g., IgG4 antibodies directed at non-CTLA-4 checkpoints (for example, anti-PD-1 IgG4 inhibitors).

In certain embodiments, the immunpotentiator is an antibody that has or is based on a human IgG1 isotype or another isotype that elicits antibody-dependent cell-mediated cytotoxicity (ADCC) and/or complement mediated cytotoxicity (CDC). In other embodiments, the immunpotentiator is an antibody that has or is based on a human IgG4 isotype or another isotype that elicits little or no antibody-dependent cell-mediated cytotoxicity (ADCC) and/or complement mediated cytotoxicity (CDC).

Exemplary PD-1-based inhibitors are described in U.S. Pat. Nos. 8,728,474, 8,952,136, and 9,073,994, and EP Patent No. 1537878B1. Exemplary anti-PD-1 antibodies include nivolumab (OPDIVO®, Bristol-Myers Squibb), pembrolizumab (KEYTRUIDA®, Merck), cemiplimab (LIBTAYO®, Regeneron/Sanofi), spartalizumab (PDR001), MEDI0680 (AMP-514), pidilizumab (CT-011), dostarlimab, sintilimab, toripalimab, camrelizumab, tislelizumab, and prolgolimab. Exemplary anti-PD-L1 antibodies include avelumab (BAVENCIO , EMD Serono/Pfizer), atezolizumab (TECENTRIQ®, Genentech), and durvalumab (IMFINZI®, Medimmune/AstraZeneca).

The PD-1-based inhibitor may be designed, expressed, and purified using techniques known to those skilled in the art, for example, as described hereinabove. The anti-PD-1 antibodies may be designed, expressed, purified, formulated and administered as described in U.S. Pat. Nos. 8,728,474, 8,952,136, and 9,073,994.

Other immunopotentiators (for example, antibodies, and various small molecules) may target signaling pathways involving one or more of the following ligands: B7-H3 (found on prostrate, renal cell, non-small cell lung, pancreatic, gastric, ovarian, colorectal cells, among others); B7-H4 (found on breast, renal cell, ovarian, pancreatic, melanoma cells, among others); HHLA2 (found on breast, lung , thyroid, melanoma, pancreas, ovary, liver, bladder, colon, prostate, kidney cells, among others); galectins (found on non-small cell lung, colorectal, and gastric cells, among others); CD30 (found on Hodgkin lymphoma, large cell lymphoma cells, among others); CD70 (found on non-Hodgkin's lymphoma, renal cells, among others); ICOSL (found on glioblastoma, melanoma cells, among others); CD155 (found on kidney, prostrate, pancreatic glioblastoma cells, among others); and TIM3. Similarly, other potential immunopotentiators that can be used include, for example, a 4-1BB (CD137) agonist (e.g., the fully human IgG4 anti-CD137 antibody Urelumab/BMS-663513), a LAG3 inhibitor (e.g., the humanized IgG4 anti-LAG3 antibody LAG525, Novartis); an IDO inhibitor (e.g., the small molecule INCB024360, Incyte Corporation), a TGFβ inhibitor (e.g., the small molecule Galunisertib, Eli Lilly) and other receptor or ligands that are found on T-cells and/or tumor cells. In certain embodiments, immunopotentiators (for example, antibodies, and various small molecules) that target signaling pathways involving one or more of the foregoing ligands are amenable to pharmaceutical intervention based on agonist/antagonist interactions but not through ADCC.

A. Antibody Production

Methods for producing antibodies are known in the art. For example, DNA molecules encoding light chain variable regions and heavy chain variable regions can be chemically synthesized using the sequences of the CDRs and variable regions of the antibodies of interest, for example, the antibody sequences provided in U.S. Pat. No. 8,952,136 and the hybridoma deposits described in U.S. Pat. No. 9,073,994. Synthetic DNA molecules can be ligated to other appropriate nucleotide sequences, including, e.g., constant region coding sequences, and expression control sequences, to produce conventional gene expression constructs encoding the desired antibodies. Production of defined gene constructs is within routine skill in the art. Alternatively, the sequences provided herein can be cloned out of hybridomas by conventional hybridization techniques or polymerase chain reaction (PCR) techniques, using synthetic nucleic acid probes whose sequences are based on sequence information provided herein, or prior art sequence information regarding genes encoding the heavy and light chains of murine antibodies in hybridoma cells.

Nucleic acids encoding the antibodies disclosed herein can be incorporated (ligated) into expression vectors, which can be introduced into host cells through conventional transfection or transformation techniques. Exemplary host cells are E. coli cells, Chinese hamster ovary (CHO) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If a DNA construct encoding an antibody disclosed herein is to be expressed in eukaryotic host cells, e.g., CHO cells, it is first inserted into an expression vector containing a suitable eukaryotic promoter, a secretion signal, IgG enhancers, and various introns. This expression vector optionally contains sequences encoding all or part of a constant region, enabling an entire, or a part of, a heavy and/or light chain to be expressed. In some embodiments, a single expression vector contains both heavy and light chain variable regions to be expressed.

The gene construct can be introduced into eukaryotic host cells using conventional techniques. The host cells express V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e.g., a heavy chain variable region) or a light chain (e.g., a light chain variable region). In other embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In still other embodiments, a host cell is co-transfected with more than one expression vector (e.g., one expression vector expressing a polypeptide comprising an entire, or part of, a heavy chain or heavy chain variable region, and another expression vector expressing a polypeptide comprising an entire, or part of, a light chain or light chain variable region).

A method of producing a polypeptide comprising an immunoglobulin heavy chain variable region or a polypeptide comprising an immunoglobulin light chain variable region may comprise growing (culturing) a host cell transfected with an expression vector under conditions that permits expression of the polypeptide comprising the immunoglobulin heavy chain variable region or the polypeptide comprising the immunoglobulin light chain variable region. The polypeptide comprising a heavy chain variable region or the polypeptide comprising the light chain variable region then may be purified using techniques well known in the art, e.g., affinity tags such as glutathione-S-transferase (GST) and histidine tags.

A method of producing a monoclonal antibody that binds a target protein, for example, PD-1, PD-L1, or PD-L2, or an antigen-binding fragment of the antibody, may comprise growing a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial chains), under conditions that permit expression of both chains. The intact antibody (or antigen-binding fragment) can be harvested and purified using techniques well known in the art, e.g., Protein A, Protein G, affinity tags such as glutathione-S-transferase (GST) and histidine tags. It is within ordinary skill in the art to express the heavy chain and the light chain from a single expression vector or from two separate expression vectors.

B. Antibody Modifications

Methods for reducing or eliminating the antigenicity of antibodies and antibody fragments are known in the art. When the antibodies are to be administered to a human, the antibodies preferably are “humanized” to reduce or eliminate antigenicity in humans. Preferably, a humanized antibody has the same or substantially the same affinity for the antigen as the non-humanized mouse antibody from which it was derived.

In one humanization approach, chimeric proteins are created in which mouse immunoglobulin constant regions are replaced with human immunoglobulin constant regions. See, e.g., Morrison et al. (1984) PROC. NAT. ACAD. SCI. 81:6851-6855, Neuberger et al. (1984) NATURE 312:604-608; U.S. Pat. No. 6,893,625 (Robinson); U.S. Pat. No. 5,500,362 (Robinson); and U.S. Pat. No. 4,816,567 (Cabilly).

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of an anti-ErbB3 antibody are grafted onto human FRs or consensus human FRs. To create consensus human FRs, FRs from several human heavy chain or light chain amino acid sequences are aligned to identify a consensus amino acid sequence. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321: 522-525; Riechmann et al. (1988) NATURE 332: 323-327; Verhoeyen et al. (1988) SCIENCE 239: 1534-1536; and Winter (1998) FEBS LETT 430: 92-94.

In an approach called “SUPERHUMANIZATION™,” human CDR sequences are chosen from human germline genes, based on the structural similarity of the human CDRs to those of the mouse antibody to be humanized. See, e.g., U.S. Pat. No. 6,881,557 (Foote); and Tan et al. (2002) J. IMMUNOL. 169:1119-1125.

Other methods to reduce immunogenicity include “reshaping,” “hyperchimerization,” and “veneering/resurfacing.” See, e.g., Vaswami et al. (1998) ANNALS OF ALLERGY, ASTHMA, & IMMUNOL. 81:105; Roguska et al. (1996) PROT. ENGINEER 9:895-904; and U.S. Pat. No. 6,072,035 (Hardman). In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, NY), which involves a vaccinia virus-based vector to express antibodies in mammalian cells. High levels of combinatorial diversity of IgG heavy and light chains are said to be produced. See, e.g., U.S. Pat. Nos. 6,706,477 (Zauderer); U.S. Pat. No. 6,800,442 (Zauderer); and U.S. Pat. No. 6,872,518 (Zauderer).

Another approach for converting a mouse antibody into a form suitable for use in humans is technology practiced commercially by KaloBios Pharmaceuticals, Inc. (Palo Alto, CA). This technology involves the use of a proprietary human “acceptor” library to produce an “epitope focused” library for antibody selection.

Another approach for modifying a mouse antibody into a form suitable for medical use in humans is HUMAN ENGINEERING™ technology, which is practiced commercially by XOMA (US) LLC. See, e.g., International (PCT) Publication No. WO 93/11794 and U.S. Pat. Nos. 5,766,886; 5,770,196; 5,821,123; and 5,869,619.

Any suitable approach, including any of the above approaches, can be used to reduce or eliminate human immunogenicity of an antibody including the binding moiety component of the superantigen conjugate disclosed herein.

Methods of making multispecific antibodies are known in the art. Multi-specific antibodies include bispecific antibodies. Bispecific antibodies are antibodies that have binding specificities for at least two different epitopes. Exemplary bispecific antibodies bind to two different epitopes of the antigen of interest. Bispecific antibodies can be prepared as full length antibodies or antibody fragments (e.g., F(ab′)₂ bispecific antibodies and diabodies) as described, for example, in Milstein et al., NATURE 305:537-539 (1983), WO 93/08829, Traunecker et al., EMBO J., 10:3655-3659 (1991), WO 94/04690, Suresh et al. (1986) METHODS IN ENZYMOLOGY 121:210, WO96/27011, Brennan et al. (1985) SCIENCE 229: 81, Shalaby et al. (1992) J. EXP. MED. 175: 217-225, Kostelny et al. (1992) J. IMMUNOL. 148(5):1547-1553, Hollinger et al. (1993) PNAS, 90:6444-6448, Gruber et al. (1994) J. IMMUNOL. 152:5368, Wu et al. (2007) NAT. BIOTECHNOL. 25(11): 1290-1297, U.S. Patent Publication No. 2007/0071675, and Bostrom et al., SCIENCE 323:1640-1644 (2009).

### IV. Formulations and Pharmaceutical Compositions

The superantigen conjugate and B-cell depleting agent, e.g., anti-CD20 antibody, can be administered to the subject so as to treat the cancer, for example, to slow the growth rate of cancer cells, reduce the incidence or number of metastases, reduce tumor size, inhibit tumor growth, reduce the blood supply to a tumor or cancer cells, promote an immune response against cancer cells or a tumor, prevent or inhibit the progression of cancer, for example, by at least 40%, 50%, 60%, 70%, 80%, 90%, 95%, 98%, 99%, or 100%. Alternatively, the superantigen conjugate and B-cell depleting agent, e.g., anti-CD20 antibody, can be administered to the subject so as to treat the cancer, for example, to increase the lifespan of a subject with cancer, for example, by 3 months, 6 months, 9 months, 12 months, 1 year, 2 years, 3 years, 4, years, 5 years, 6 years, 7 years, 8 years, 9 years, or 10 years. Alternatively, the superantigen conjugate and B-cell depleting agent, e.g., anti-CD20 antibody, can be administered to the subject so as to treat the cancer, for example, to facilitate cancer free survival of a subject following cancer treatment, for example, for 3 months, 6 months, 9 months, 12 months, 1 year, 2 years, 3 years, 4 years, 5 years, 6 years, 7 years, 8 years, 9 years, or 10 years. Alternatively, the superantigen conjugate and B-cell depleting agent, e.g., anti-CD20 antibody, can be administered to the subject so as to treat the cancer, for example, to prevent cancer progression in a subject following cancer treatment, for example, for 3 months, 6 months, 9 months, 12 months, 1 year, 2 years, 3 years, 4 years, 5 years, 6 years, 7 years, 8 years, 9 years, or 10 years. In each approach, the superantigen conjugate and B-cell depleting agent, e.g., anti-CD20 antibody, can be administered together, sequentially, or intermittently to the subject.

The superantigen conjugate and B-cell depleting agent, e.g., anti-CD20 antibody, may be formulated separately or together using techniques known to those skilled in the art. For example, for therapeutic use, the superantigen conjugate and/or B-cell depleting agent, e.g., anti-CD20 antibody, is combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

The superantigen conjugate is used in combination with B-cell depleting agent. The superantigen conjugate can be administered separately or simultaneously (in the same or different formulations) with B-cell depleting agent. In some embodiments, the B-cell depleting agent is administered prior to the superantigen conjugate.

Pharmaceutical compositions containing the superantigen conjugate and/or B-cell depleting agent, e.g., anti-CD20 antibody, disclosed herein can be provided in a single dosage form or different dosage forms. The pharmaceutical composition or compositions should be formulated to be compatible with its intended route of administration. Examples of routes of administration are intravenous (IV), intramuscular, intradermal, inhalation, transdermal, topical, transmucosal, and rectal administration. Alternatively, the agents may be administered locally rather than systemically, for example, via injection of the agent or agents directly into the site of action, often in a depot or sustained release formulation.

Useful formulations can be prepared by methods well known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

The superantigen conjugate and/or B-cell depleting agent, e.g., anti-CD20 antibody, of the present invention may be employed alone or in conjunction with other compounds, such as carriers or other therapeutic compounds. Pharmaceutical compositions of the present invention comprise an effective amount of one or more superantigen conjugates and optionally one or more B-cell depleting agents, e.g., anti-CD20 antibodies, and may also contain additional agents, dissolved or dispersed in a pharmaceutically acceptable carrier. The phrases “pharmaceutical” or “pharmacologically acceptable” refer to substances, e.g., compositions, that do not produce an adverse, allergic or other untoward reaction when administered to a mammal, such as, for example, a human. The preparation of a pharmaceutical composition that contains at least one superantigen conjugate and/or B-cell depleting agent, e.g., anti-CD20 antibody, will be known to those of skill in the art in light of the present disclosure, and as exemplified by Remington's Pharmaceutical Sciences, 18th Ed. Mack Printing Company, 1990, incorporated herein by reference. Moreover, for human administration, it will be understood that preparations should meet sterility, pyrogenicity, general safety and purity standards as required by FDA Office of Biological Standards.

In a specific embodiment of the invention, the compositions of the invention comprise tumor-targeted superantigen in combination with B-cell depleting agent, e.g., anti-CD20 antibody. Such combinations include, for example, any tumor-targeted superantigen and/or B-cell depleting agent, e.g., anti-CD20 antibody, as described herein.

In a specific embodiment of the invention, the tumor-targeted super antigen comprises a bacterial superantigen including, but are not limited to, Staphylococcal enterotoxin (SE), Streptococcus pyogenes exotoxin (SPE), Staphylococcus aureus toxic shock-syndrome toxin (TSST-1), Streptococcal mitogenic exotoxin (SME), Streptococcal superantigen (SSA), Staphylococcal enterotoxin A (SEA), Staphylococcal enterotoxin B (SEB), and Staphylococcal enterotoxin E (SEE) conjugated to a targeting moiety. In another embodiment of the invention, the compositions comprise tumor-targeted superantigens comprising superantigens with the following Protein Data Bank and/or GenBank accession numbers include, but are not limited to, SEE is P12993; SEA is P013163; SEB is P01552; SEC1 is P01553; SED is P20723; and SEH is AAA19777, as well as variants thereof, conjugated to a targeting moiety.

In certain embodiments, the superantigen conjugate comprises a wild type or engineered superantigen sequence such as, the wild-type SEE sequence (SEQ ID NO: 1) or the wild type SEA sequence (SEQ ID NO: 2), either of which can be modified such that amino acids in any of the identified regions A-E (see, FIG. 1) are substituted with other amino acids. In certain embodiments, the superantigen incorporated in the conjugate is SEA/E-120 (SEQ ID NO: 3) or SEAD227A (SEQ ID NO: 4).

Specific examples of targeting moieties to be conjugated to the superantigens include, for example, any molecule that is able to bind to a cellular molecule and preferably a disease specific molecule such as a cancer cell specific molecule. The targeting moiety can be selected from antibodies, including antigen binding fragments, soluble T-cell receptors, growth factors, interleukins, hormones, etc. Exemplary cancer targeting antibodies can include, but are not limited to, anti-CD19, anti-CD20 antibodies, anti-5T4 antibodies, anti-Ep-CAM antibodies, anti-Her-2/neu antibodies, anti-EGFR antibodies, anti-CEA antibodies, anti-prostate specific membrane antigen (PSMA) antibodies, and anti-IGF-1R antibodies. In one embodiment, the superantigen can be conjugated to an immunologically reactive antibody fragment such as C215Fab, 5T4Fab (see, WO8907947) or C242Fab (see, WO9301303).

Examples of such tumor-targeted superantigens include C215Fab-SEA (SEQ ID NO: 5), 5T4Fab-SEA_{D227A} (SEQ ID NO: 6) and 5T4Fab-SEA/E-120 (SEQ ID NO: 7). In a preferred embodiment, the superantigen conjugate is 5T4 Fab-SEA/E-120, known in the art as naptumomab estafenatox, which comprises two polypeptide sequences that together define an Fab fragment of an anti-5T4 antibody, where one of the polypeptide sequences further comprises the SEA/E-120 superantigen namely SEQ ID NO: 8 (chimeric V_{H} chain of 5T4 Fab coupled by three amino acid linker to SEA/E-120) and SEQ ID NO: 9 (chimeric V_{L} chain of 5T4 Fab).

In an exemplary embodiment, the compositions of the invention comprise the tumor-targeted superantigen 5T4Fab-SEA/E-120, known in the art as naptumomab estafenatox in combination with anti-CD20 antibody, e.g., ibritumomab, obinutuzumab, ocaratuzumab, ocrelizumab, ofatumumab, rituximab, veltuzumab, tositumomab, ublituximab, veltuzumab, PRO131921, or TRU-015, e.g., obinutuzumab.

Formulations or dosage form containing the superantigen conjugate and B-cell depleting agent, e.g., anti-CD20 antibody, may comprise different types of carriers depending on whether they are to be administered in solid, liquid or aerosol form, and whether it need to be sterile for such routes of administration as injection.

Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In certain embodiments, pharmaceutical compositions may comprise, for example, at least about 0.1% of an active compound. In other embodiments, the active compound may comprise between about 2% to about 75% of the weight of the unit, or between about 25% to about 60%, for example, and any range derivable therein. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable. Such determinations are known and used by those of skill in the art.

The active agents are administered in an amount or amounts effective to decrease, reduce, inhibit or otherwise abrogate the growth or proliferation of cancer cells, induce apoptosis, inhibit angiogenesis of a cancer or tumor, inhibit metastasis, or induce cytotoxicity in cells. The effective amount of active compound(s) used to practice the present invention for therapeutic treatment of cancer varies depending upon the manner of administration, the age, body weight, and general health of the subject. These terms include synergistic situations such as those presented and described in the instant invention wherein a single agent alone, such as a superantigen conjugate or B-cell depleting agent, e.g., anti-CD20 antibody, may act weakly or not at all, but when combined with each other, for example, but not limited to, via sequential dosage, the two or more agents act to produce a synergistic result.

In certain non-limiting examples, a dose of the superantigen conjugate may comprise from about 1 microgram/kg/body weight, about 5 microgram/kg/body weight, about 10 microgram/kg/body weight, about 15 microgram/kg/body weight, about 20 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, about 1 microgram/kg/body weight to about 100 milligram/kg/body weight. Other exemplary dosage ranges, range from about 1 microgram/kg/body weight to about 1000 microgram/kg/body weight, from about 1 microgram/kg/body weight to about 100 microgram/kg/body weight, from about 1 microgram/kg/body weight to about 75 microgram/kg/body weight, from about 1 microgram/kg/body weight to about 50 microgram/kg/body weight, from about 1 microgram/kg/body weight to about 40 microgram/kg/body weight, from about 1 microgram/kg/body weight to about 30 microgram/kg/body weight, from about 1 microgram/kg/body weight to about 20 microgram/kg/body weight, from about 1 microgram/kg/body weight to about 15 microgram/kg/body weight, from about 1 microgram/kg/body weight to about 10 microgram/kg/body weight, from about 5 microgram/kg/body weight to about 1000 microgram/kg/body weight, from about 5 microgram/kg/body weight to about 100 microgram/kg/body weight, from about 5 microgram/kg/body weight to about 75 microgram/kg/body weight, from about 5 microgram/kg/body weight to about 50 microgram/kg/body weight, from about 5 microgram/kg/body weight to about 40 microgram/kg/body weight, from about 5 microgram/kg/body weight to about 30 microgram/kg/body weight, from about 5 microgram/kg/body weight to about 20 microgram/kg/body weight, from about 5 microgram/kg/body weight to about 15 microgram/kg/body weight, from about 5 microgram/kg/body weight to about 10 microgram/kg/body weight, from about 10 microgram/kg/body weight to about 1000 microgram/kg/body weight, from about 10 microgram/kg/body weight to about 100 microgram/kg/body weight, from about 10 microgram/kg/body weight to about 75 microgram/kg/body weight, from about 10 microgram/kg/body weight to about 50 microgram/kg/body weight, from about 10 microgram/kg/body weight to about 40 microgram/kg/body weight, from about 10 microgram/kg/body weight to about 30 microgram/kg/body weight, from about 10 microgram/kg/body weight to about 20 microgram/kg/body weight, from about 10 microgram/kg/body weight to about 15 microgram/kg/body weight, from about 15 microgram/kg/body weight to about 1000 microgram/kg/body weight, from about 15 microgram/kg/body weight to about 100 microgram/kg/body weight, from about 15 microgram/kg/body weight to about 75 microgram/kg/body weight, from about 15 microgram/kg/body weight to about 50 microgram/kg/body weight, from about 15 microgram/kg/body weight to about 40 microgram/kg/body weight, from about 15 microgram/kg/body weight to about 30 microgram/kg/body weight, from about 15 microgram/kg/body weight to about 20 microgram/kg/body weight, from about 20 microgram/kg/body weight to about 1000 microgram/kg/body weight, from about 20 microgram/kg/body weight to about 100 microgram/kg/body weight, from about 20 microgram/kg/body weight to about 75 microgram/kg/body weight, from about 20 microgram/kg/body weight to about 50 microgram/kg/body weight, from about 20 microgram/kg/body weight to about 40 microgram/kg/body weight, from about 20 microgram/kg/body weight to about 30 microgram/kg/body weight, etc., can be administered, based on the numbers described above.

In certain embodiments, the effective amount or dose of the superantigen conjugate that is administered is an amount in the range of 0.01 to 500 μg/kg body weight of the subject, for example, 0.1-500 μg/kg body weight of the subject, and, for example, 1-100 μg/kg body weight of the subject.

In certain embodiments, the effective amount or dose of the B-cell depleting agent is an amount or dose that effectively reduces the number of B-cells in the subject. In certain embodiments, the effective amount or dose of the B-cell depleting agent is an amount or dose that effectively reduces the number of B-cells in the subject prior to administration of the superantigen conjugate. In certain embodiments, the effective amount or dose of the B-cell depleting agent, e.g., anti-CD20 antibody, that is administered in combination with the superantigen conjugate is an amount or dose that results in at least an additive or a synergistic anti-tumor effect.

Generally, a therapeutically effective amount of a B-cell depleting agent, e.g., anti-CD20 antibody, is in the range of 0.1 mg/kg to 100 mg/kg, e.g., 1 mg/kg to 100 mg/kg or 1 mg/kg to 10 mg/kg. In certain embodiments, the effective amount or dose of the B-cell depleting agent (e.g., anti-CD20 antibody) is about 2 g (for example, administered as a single administration of about 2 g, or as several administrations, e.g., two administrations of about 1 g each or three administrations of, e.g., 100 mg, 900 mg and 1000 mg). In certain embodiments, the effective amount or dose of the B-cell depleting agent (e.g., anti-CD20 antibody) is about 1000 mg (for example, administered as a single administration of about 1000 mg, or as several administrations, e.g., two administrations of about 500 mg each).

The amount of B-cell depleting agent, e.g., anti-CD20 antibody, administered will depend on variables such as the type and extent of disease or indication to be treated, the overall health of the patient, the in vivo potency of the superantigen conjugate and the B-cell depleting agent, the pharmaceutical formulation, and the route of administration.

### V. Treatment Regimens and Indications

Treatment regimens may vary as well, and often depend on tumor type, tumor location, disease progression, and health and age of the patient. Certain types of tumor may require more aggressive treatment protocols, but at the same time, the patients may be unable to tolerate more aggressive treatment regimens. The clinician may often be best suited to make such decisions based on his or her skill in the art and the known efficacy and toxicity (if any) of the therapeutic formulations.

In a specific embodiment of the invention, the treatment methods of the invention comprise administration of a tumor-targeted superantigen, in combination with B-cell depleting agent, e.g., anti-CD20 antibody, to a patient in need thereof, i.e., a cancer patient. Such combination treatments include, for example, administration of any tumor-targeted superantigen and/or B-cell depleting agent, e.g., anti-CD20 antibody, as described herein. In a specific embodiment of the invention, the tumor-targeted super antigen comprises a bacterial superantigen including, but are not limited to, Staphylococcal enterotoxin (SE), Streptococcus pyogenes exotoxin (SPE), Staphylococcus aureus toxic shock-syndrome toxin (TSST-1), Streptococcal mitogenic exotoxin (SME), Streptococcal superantigen (SSA), Staphylococcal enterotoxin A (SEA), Staphylococcal enterotoxin B (SEB), and Staphylococcal enterotoxin E (SEE) conjugated to a targeting moiety.

In certain embodiments, the superantigen conjugate comprises a wild type or engineered superantigen sequence such as, the wild-type SEE sequence (SEQ ID NO: 1) or the wild type SEA sequence (SEQ ID NO: 2), either of which can be modified such that amino acids in any of the identified regions A-E (see, FIG. 1) are substituted with other amino acids. In certain embodiments, the superantigen incorporated in the conjugate is SEA/E-120 (SEQ ID NO: 3) or SEAD227A (SEQ ID NO: 4).

Specific examples of targeting moieties to be conjugated to the superantigens include, for example, any molecule that is able to bind to a cellular molecule and preferably a disease specific molecule such as a cancer cell specific molecule. The targeting moiety can be selected from antibodies, including antigen binding fragments, soluble T-cell receptors, growth factors, interleukins, hormones, etc. Exemplary cancer targeting antibodies can include, but are not limited to, anti-CD19, anti-CD20 antibodies, anti-5T4 antibodies, anti-Ep-CAM antibodies, anti-Her-2/neu antibodies, anti-EGFR antibodies, anti-CEA antibodies, anti-prostate specific membrane antigen (PSMA) antibodies, and anti-IGF-1R antibodies. In one embodiment, the superantigen can be conjugated to an immunologically reactive antibody fragment such as C215Fab, 5T4Fab (see, WO8907947) or C242Fab (see, WO9301303).

Examples of such tumor-targeted superantigens include C215Fab-SEA (SEQ ID NO: 5), 5T4Fab-SEA_{D227A} (SEQ ID NO: 6) and 5T4Fab-SEA/E-120 (SEQ ID NO: 7). In a preferred embodiment, the superantigen conjugate is 5T4 Fab-SEA/E-120 known in the art as naptumomab estafenatox, which comprises two polypeptide sequences that together define an Fab fragment of an anti-5T4 antibody, where one of the polypeptide sequences further comprises the SEA/E-120 superantigen namely SEQ ID. NO: 8 (chimeric V_{H} chain of 5T4 Fab coupled by three amino acid linker to SEA/E-120) and SEQ ID. NO: 9 (chimeric V_{L} chain of 5T4 Fab).

In a preferred embodiment, the compositions of the invention comprise the tumor-targeted superantigen 5T4Fab-SEA/E-120, known in the art as naptumomab estafenatox optionally in combination with a B-cell depleting agent, e.g., anti-CD20 antibody, e.g., ibritumomab, obinutuzumab, ocaratuzumab, ocrelizumab, ofatumumab, rituximab, veltuzumab, tositumomab, ublituximab, veltuzumab, PRO131921, or TRU-015, e.g., obinutuzumab.

Preferably, patients to be treated will have adequate bone marrow function (defined as a peripheral absolute granulocyte count of >2,000/mm³ and a platelet count of 100,000/mm³), adequate liver function (bilirubin<1.5 mg/dl) and adequate renal function (creatinine<1.5 mg/dl).

A typical course of treatment may involve multiple doses. Typical treatment may involve a 6 dose application over a two-week period. The two-week regimen may be repeated one, two, three, four, five, six or more times. During a course of treatment, the need to complete the planned dosings may be re-evaluated.

Immunotherapy with the superantigen conjugate often results in rapid (within hours) and powerful polyclonal activation of T lymphocytes. A superantigen conjugate treatment cycle may include 4 to 5 daily intravenous superantigen conjugate drug injections. Such treatment cycles can be given in e.g., 3 to 8 week intervals. The inflammation with infiltration of CTLs into the tumor is one of the major effectors of the anti-tumor therapeutic superantigens. After a short period of massive activation and differentiation of CTLs, the T-cell response declines rapidly (within 4-5 days) back to base line levels. Thus, the period of lymphocyte proliferation, during which cytostatic drugs may interfere with superantigen treatment is short and well-defined.

In certain embodiments, the treatment regimen of the present invention may involve administering the superantigen conjugate and the B-cell depleting agent, e.g., anti-CD20 antibody, to the subject at the same time. This may be achieved by administering to the subject a single composition or pharmacological formulation that includes both agents, or by administering to the subject two distinct compositions or formulations, at the same time, wherein one composition includes the superantigen conjugate and the other includes the B-cell depleting agent, e.g., anti-CD20 antibody.

Alternatively, the superantigen conjugate may precede or follow the B-cell depleting agent, e.g., anti-CD20 antibody, by intervals ranging from minutes, days to weeks. In embodiments where B-cell depleting agent, e.g., anti-CD20 antibody, and the superantigen conjugate are administered separately to the subject, one should ensure that a significant period of time does not expire between the time of each delivery, such that the superantigen conjugate and B-cell depleting agent, e.g., anti-CD20 antibody, would still be able to exert an advantageously combined effect on the subject. In some situations, it may be desirable to treat the subject with both modalities within about 12-72 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, the superantigen conjugate being “A” and the B-cell depleting agent, e.g., anti-CD20 antibody, being “B”: AB/A, B/A/B, BB/A, A/A/B, A/B/B, B/A/A, A/BBB, B/A/B/B, BBB/A, B/B/A/B, A/A/B/B, AB/AB, A/B/B/A, B/B/A/A, B/A/B/A, B/A/A/B, A/A/A/B, B/A/A/A, A/B/A/A, and A/A/B/A.

In certain embodiments, a contemplated method comprises first administering to the subject an effective amount of a B-cell depleting agent, e.g., an anti-CD20 antibody, e.g., an anti-CD20 antibody contemplated herein, and then administering to the subject an effective amount of a superantigen conjugate, e.g., a superantigen conjugate contemplated herein. In certain embodiments, the period of time between the administration of the B-cell depleting agent and the administration of the superantigen conjugate is a period of time that effectively reduces the number of B-cells in the subject prior to administration of the superantigen conjugate. It is contemplated that the B-cell depleting agent may be administered to a subject once, twice, or more than twice. In certain embodiments, when a subject receives two or more than two administrations of the B-cell depleting agent, the two or more than two administrations may be on two or more consecutive days.

For example, in certain embodiments, the subject receives an administration (e.g., the initial administration) of the B-cell depleting agent at least about 1, 2, 3, 4, 5, 6, 7, 10, 11, 12, 13, 14, 15, 21, or 28 days prior to an administration (e.g., the initial administration) of the superantigen conjugate. In certain embodiments, the subject receives an administration (e.g., the initial administration) of the B-cell depleting agent about 1, 2, 3, 4, 5, 6, 7, 10, 11, 12, 13, 14, 15, 21, or 28 days, or greater than 28 days, prior to an administration (e.g., the initial administration) of the superantigen conjugate. In certain embodiments, the subject receives an administration (e.g., the initial administration) of the B-cell depleting agent from about 1 to about 28, from about 1 to about 21, from about 1 to about 14, from about 1 to about 7, from about 7 to about 28, from about 7 to about 21, from about 7 to about 14, from about 14 to about 28, from about 14 to about 21, or from about 21 to about 28 days prior to an administration (e.g., the initial administration) of the superantigen conjugate. In certain embodiments, the subject receives an administration of the B-cell depleting agent 1, 2, 3, 4, 5, 6, 7, 10, 11, 12, 13, 14, 15, 21, and/or 28 days prior to an administration (e.g., the initial administration) of the superantigen conjugate. For example, the subject may receive an administration of the B-cell depleting agent 12 and 13 days prior to an administration (e.g., the initial administration) of the superantigen conjugate (e.g., at a dose of 1000 mg B-cell depleting agent per day).

In certain embodiments, a subject is administered a B-cell depleting agent, e.g., an anti-CD20 antibody, e.g., an anti-CD20 antibody contemplated herein, on days 1, 2, 8 and 15 of a first 28 day treatment cycle (for example, at a dose of 100 mg on day 1, 900 mg on day 2, 1000 mg on day 8, and 1000 mg on day 15). In certain embodiments, the subject is administered the B-cell depleting agent on day 1 of optional subsequent treatment cycles (e.g., at a dose of 1000 mg on day 1).

In certain embodiments, a subject is administered (i) a superantigen conjugate, e.g., a superantigen conjugate contemplated herein, daily for 2 to 6 consecutive days (e.g., 2, 3, 4, 5, or 6 consecutive days) every 2 to 12 weeks (e.g., 2, 3, 4, 5, 6, 7, 8, 9, 10, 11, or 12 weeks), and/or (ii) a B-cell depleting agent, e.g., an anti-CD20 antibody, e.g., an anti-CD20 antibody contemplated herein, 13 and 12 days prior to an administration (e.g., the initial administration) of the superantigen conjugate.

In certain embodiments, administration of the B-cell depleting agent effectively reduces the formation of anti-drug antibodies (ADAs) in the subject in response to the administration of the superantigen conjugate as compared to a corresponding method without the administration of the B-cell depleting agent.

An “anti-drug antibody” or “ADA” refers to an antibody that binds to a therapeutic agent, e.g., a superantigen conjugate, and may influence serum concentrations and function of the therapeutic agent in a subject. The presence of ADAs may increase clearance of the therapeutic agent through formation of immune complexes between the therapeutic agent and the antibody (neutralizing, non-neutralizing or both), thus reducing the therapeutic agent's half-life. Furthermore, the activity and efficacy of the therapeutic agent may be decreased through binding of the antibody to the therapeutic agent (particularly in the case of neutralizing ADAs). ADAs can also be associated with allergic or hypersensitivity reactions and other adverse events.

In certain embodiments, a contemplated method effectively reduces the formation of anti-drug antibodies (ADAs) in a subject in response to the administration of the superantigen conjugate as compared to a corresponding method without the administration of the B-cell depleting agent, e.g., anti-CD20 antibody. In certain embodiments, the formation of ADAs is reduced at least 2-fold, at least 3-fold, at least 4-fold, at least 5-fold, at least 10-fold, at least 20-fold, at least 50-fold, or at least 100-fold as compared to a corresponding method without the administration of the B-cell depleting agent, e.g., anti-CD20 antibody. In certain embodiments, the formation of ADAs is essentially prevented. In certain embodiments, the reduction or prevention of the formation of ADAs is for at least 1, 2, 3, 4, 5, 6, 7, 8, 9, 10, 11, 12, 13, 14, 15, 16, 17, 18, 19, 20 or 21 days, or 1 month, 2 months, 3 months, 4 months, 5 months, 6 months, 12 months, or more, after administration of the superantigen conjugate. In certain embodiments, the reduction or prevention of ADA is for about 2 months after administration of the superantigen conjugate.

In certain embodiments, the ADA titer in the subject after administration of the superantigen conjugate does not exceed the ADA titer in the subject prior to administration of the superantigen conjugate. In certain embodiments, the ADA titer in the subject after administration of the superantigen conjugate does not exceed the ADA titer in the subject prior to administration of the superantigen conjugate by more than 1.1-fold, more than 1.2-fold, more than 1.5-fold, more than 2-fold, more than 3-fold, more than 4-fold, more than 5-fold, or more than 10-fold. In certain embodiments, the ADA titer in the subject after administration of the superantigen conjugate is increased less than 1.1-fold, less than 1.2-fold, less than 1.5-fold, less than 2-fold, less than 3-fold, less than 4-fold, less than 5-fold, or less than 10-fold, as compared to the ADA titer in the subject prior to administration of the superantigen conjugate. In certain embodiments, the ADA titer in the subject after administration of the superantigen conjugate is the ADA titer at 1, 2, 3, 4, 5, 6, 7, 8, 9, 10, 11, 12, 13, 14, 15, 16, 17, 18, 19, 20 or 21 days, or 1 month, 2 months, 3 months, 4 months, 5 months, 6 months, 12 months, or more, after administration of the superantigen conjugate. In certain embodiments, the ADA titer in the subject after administration of the superantigen conjugate is the ADA titer at about 2 months after administration of the superantigen conjugate.

In certain embodiments, essentially no ADAs are detectable in the subject after administration of the superantigen conjugate, for example, at 1, 2, 3, 4, 5, 6, 7, 8, 9, 10, 11, 12, 13, 14, 15, 16, 17, 18, 19, 20 or 21 days, or 1 month, 2 months, 3 months, 4 months, 5 months, 6 months, 12 months, or more, after administration of the superantigen conjugate.

ADAs can be detected, for example, in a blood sample taken from the subject. ADAs can be detected by any method known in the art. Exemplary methods to detect ADAs are described in Mire-Sluis et al. (2004) J. IMMUNOL. METHODS 289:1-16, Nencini et al. (2014) DRUG DEV. RES. 75 Suppl 1:S4-6, and Schouwenburg et al. (2015) NAT. REV. RHEUMATOL. 9, 164-172. An exemplary method to detect ADAs is a sandwich ELISA, in which the superantigen conjugate is coated to an assay plate, is exposed to serum of the treated subject, and the presence of ADAs is detected by labelled superantigen conjugate. Another exemplary method to detect ADAs is an antigen binding test wherein immunoglobulins from the treated subject's serum are aggregated on a protein (e.g. Protein A Sepharose) and the presence of ADAs is detected by labelled superantigen conjugate.

Furthermore, the superantigen conjugate and/or B-cell depleting agent, e.g., anti-CD20 antibody, may be co-administered together or sequentially with one or more additional agents that enhance the potency and/or selectively of the therapeutic effect. Such agents include, for example, corticosteroids, additional immune modulators, and compounds designed to reduce the patient's possible immunoreactivity to the administered superantigen conjugate.

In certain embodiments, the superantigen conjugate and/or B-cell depleting agent, e.g., anti-CD20 antibody, may be co-administered together or sequentially with a chemotherapeutic agent. Exemplary chemotherapeutic agents include antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. Additional exemplary chemotherapeutic agents include a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

It is further envisioned that the present invention can be used in combination with surgical intervention. In the case of surgical intervention, the present invention may be used preoperatively, e.g., to render an inoperable tumor subject to resection. Alternatively, the present invention may be used at the time of surgery, and/or thereafter, to treat residual or metastatic disease. For example, a resected tumor bed may be injected or perfused with a formulation comprising the tumor-targeted superantigen and/or B-cell depleting agent, e.g., anti-CD20 antibody. The perfusion may be continued post-resection, for example, by leaving a catheter implanted at the site of the surgery. Periodic post-surgical treatment also is envisioned. Any combination of the invention therapy with surgery is within the scope of the invention.

Continuous administration also may be applied where appropriate, for example, where a tumor is excised and the tumor bed is treated to eliminate residual, microscopic disease. Delivery via syringe or cauterization is preferred. Such continuous perfusion may take place for a period from about 1-2 hours, to about 2-6 hours, to about 6-12 hours, to about 12-24 hours, to about 1-2 days, to about 1-2 weeks or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

It is contemplated that a number of cancers may be treated using the methods and compositions described herein, including but not limited to primary or metastatic melanoma, adenocarcinoma, squamous cell carcinoma, adenosquamous cell carcinoma, thymoma, lymphoma, sarcoma, lung cancer, liver cancer, non-Hodgkin's lymphoma, Hodgkin's lymphoma, leukemia, uterine cancer, breast cancer, prostate cancer, ovarian cancer, pancreatic cancer, colon cancer, multiple myeloma, neuroblastoma, NPC, bladder cancer, cervical cancer and the like.

Moreover, the cancer that may be treated using the methods and compositions described herein may be based upon the body location and/or system to be treated, for example, but not limited to bone (e.g., Ewing's Family of tumors, osteosarcoma); brain (e.g., adult brain tumor, (e.g., adult brain tumor, brain stem glioma (childhood), cerebellar astrocytoma (childhood), cerebral astrocytoma/malignant glioma (childhood), ependymoma (childhood), medulloblastoma (childhood), supratentorial primitive neuroectodermal tumors and pineoblastoma (childhood), visual pathway and hypothalamic glioma (childhood) and childhood brain tumor (other)); breast (e.g., female or male breast cancer); digestive/gastrointestinal (e.g., anal cancer, bile duct cancer (extrahepatic), carcinoid tumor (gastrointestinal), colon cancer, esophageal cancer, gallbladder cancer, liver cancer (adult primary), liver cancer (childhood), pancreatic cancer, small intestine cancer, stomach (gastric) cancer); endocrine (e.g., adrenocortical carcinoma, carcinoid tumor (gastrointestinal), islet cell carcinoma (endocrine pancreas), parathyroid cancer, pheochromocytoma, pituitary tumor, thyroid cancer); eye (e.g., melanoma (intraocular), retinoblastoma); genitourinary (e.g., bladder cancer, kidney (renal cell) cancer, penile cancer, prostate cancer, renal pelvis and ureter cancer (transitional cell), testicular cancer, urethral cancer, Wilms' Tumor and other childhood kidney tumors); germ cell (e.g., extracranial germ cell tumor (childhood), extragonadal germ cell tumor, ovarian germ cell tumor, testicular cancer); gynecologic (e.g., cervical cancer, endometrial cancer, gestational trophoblastic tumor, ovarian epithelial cancer, ovarian germ cell tumor, ovarian low malignant potential tumor, uterine sarcoma, vaginal cancer, vulvar cancer); head and neck (e.g., hypopharyngeal cancer, laryngeal cancer, lip and oral cavity cancer, metastatic squamous neck cancer with occult primary, nasopharyngeal cancer, oropharyngeal cancer, paranasal sinus and nasal cavity cancer, parathyroid cancer, salivary gland cancer); lung (e.g., non-small cell lung cancer, small cell lung cancer); lymphoma (e.g., AIDS-Related Lymphoma, cutaneous T-cell lymphoma, Hodgkin's Lymphoma (adult), Hodgkin's Lymphoma (childhood), Hodgkin's Lymphoma during pregnancy, mycosis fungoides, Non-Hodgkin's Lymphoma (adult), Non-Hodgkin's Lymphoma (childhood), Non-Hodgkin's Lymphoma during pregnancy, primary central nervous system lymphoma, Sezary Syndrome, T-cell lymphoma (cutaneous), Waldenstrom's Macroglobulinemia); musculoskeletal (e.g., Ewing's Family of tumors, osteosarcoma/malignant fibrous histiocytoma of bone, rhabdomyosarcoma (childhood), soft tissue sarcoma (adult), soft tissue sarcoma (childhood), uterine sarcoma); neurologic (e.g., adult brain tumor, childhood brain tumor (e.g., brain stem glioma, cerebellar astrocytoma, cerebral astrocytoma/malignant glioma, ependymoma, medulloblastoma, supratentorial primitive neuroectodermal tumors and pineoblastoma, visual pathway and hypothalamic glioma, other brain tumors), neuroblastoma, pituitary tumor primary central nervous system lymphoma); respiratory/thoracic (e.g., non-small cell lung cancer, small cell lung cancer, malignant mesothelioma, thymoma and thymic carcinoma); and skin (e.g., cutaneous T-cell lymphoma, Kaposi's sarcoma, melanoma, and skin cancer).

It is understood that the combination of superantigen conjugate and B-cell depleting agent can be used to treat a variety of cancers, for example, a cancer selected from breast cancer, bladder cancer, cervical cancer, colon cancer, colorectal cancer, endometrial cancer, gastric cancer, head and neck cancer, liver cancer, melanoma, mesothelioma, non-small cell lung cancer, ovarian cancer, pancreatic cancer, prostate cancer, renal cell cancer, and skin cancer. In certain embodiments, the method can be used to treat head and neck cancer (e.g., in combination with atezolizumab).

Yet further, the cancer may include a tumor comprised of tumor cells. For example, tumor cells may include, but are not limited to melanoma cell, a bladder cancer cell, a breast cancer cell, a lung cancer cell, a colon cancer cell, a prostate cancer cell, a liver cancer cell, a pancreatic cancer cell, a stomach cancer cell, a testicular cancer cell, a renal cancer cell, an ovarian cancer cell, a lymphatic cancer cell, a skin cancer cell, a brain cancer cell, a bone cancer cell, or a soft tissue cancer cell. Examples of solid tumors that can be treated according to the invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In certain embodiments, the combination of superantigen conjugate and B-cell depleting agent can be used to treat a hematopoietic cancer. Exemplary hematopoietic cancers include leukemia, acute leukemia, acute lymphoblastic leukemia (ALL; e.g., B-cell ALL, T-cell ALL, or pre-B ALL), FAB ALL, acute myeloid leukemia (AML), chronic myelocytic leukemia (CML), chronic lymphocytic leukemia (CLL; e.g., transformed CLL), diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation).

In certain embodiments, the combination of superantigen conjugate and B-cell depleting agent can be used to treat chronic lymphocytic leukemia (e.g., relapsed or refractory chronic lymphocytic leukemia; e.g., in combination with chlorambucil, CC-99282, ibrutinib, rIL-15, TGR-1202, FT596, CAL-101, ABT-199, TRU-016, rituximab, entospletinib, tirabrutinib, and/or venetoclax (ABT-199)), Mantle cell lymphoma (e.g., in combination with chlorambucil), follicular lymphoma (e.g., in combination with bendamustine, CHOP, CVP, mosunetuzumab, INCB050465, or BGB3111), post-transplant lymphoproliferative disorder, acute lymphocytic leukemia (ALL; e.g., in combination with idelalisib), primary CNS lymphoma (e.g., in combination with venetoclax), Richter's transformation or Richter's syndrome (e.g., in combination with atezolizumab, venetoclax, methylprednisolone (e.g., high dose methylprednisolone (HDMP)), and/or lenalidomide), small lymphocytic lymphoma (e.g., in combination with CC-99282 or entospletinib), Waldenstrom macroglobulinemia, non-Hodgkin's lymphoma (NHL; e.g., indolent NHL; e.g., in combination with CC-122, entospletinib, glofitamab, DCDS0780A, RO7227166, and/or tocilizumab), diffuse large B-cell lymphoma (DLBCL; e.g., relapsed and/or refractory DLBCL; e.g., in combination with CC-122 or polatuzumab vedotin), or peripheral T-cell lymphomas (PTCL; e.g., in combination with PCTL).

### VI. Kits

In addition, the invention provides kits comprising, for example, a first container containing a superantigen conjugate and a second container containing a B-cell depleting agent, e.g., anti-CD20 antibody. Such a kit may also contain additional agents such as, for example, corticosteroid or another lipid modulator. The container means may itself be a syringe, pipette, and/or other such like apparatus, from which the formulation may be applied to a specific area of the body, injected into an animal, and/or applied and/or mixed with the other components of the kit.

The kits may comprise a suitably aliquoted superantigen conjugate and/or B-cell depleting agent, e.g., anti-CD20 antibody, and optionally, lipid and/or additional agent compositions of the present invention. The components of the kits may be packaged either in aqueous media or in lyophilized form. When the components of the kit are provided in one and/or more liquid solutions, the liquid solution is a sterile aqueous solution.

Practice of the invention will be more fully understood from the foregoing examples, which are presented herein for illustrative purposes only, and should not be construed as limiting the invention in any way.

### EXAMPLES

### Example 1: Tumor Targeted Superantigen and B-cell Depleting Agent Therapy

This Example describes an in vivo study testing a tumor targeted superantigen in combination with a B-cell depleting agent (an anti-CD20 antibody) in a mouse colon cancer model.

Mice were treated with either: (i) IgG control, (ii) anti-CD20 antibody, (iii) tumor targeted superantigen, (iv) anti-CD20 antibody and tumor targeted superantigen, (v) anti-PD-L1 antibody, or (vi) anti-CD20 antibody and anti-PD-L1 antibody.

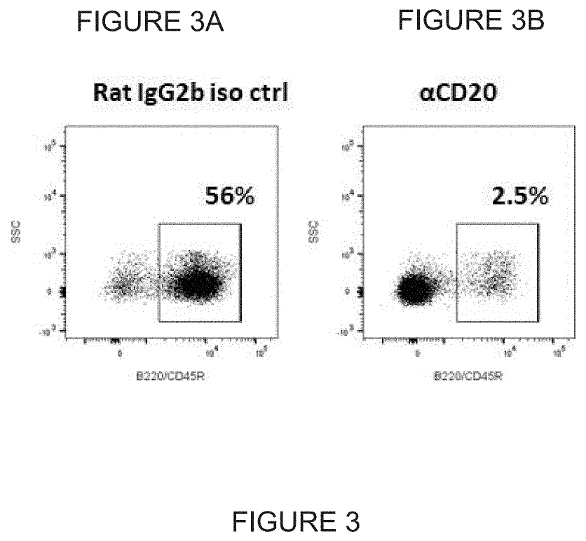

In brief, on day -7, mice were injected i.v. with 250 μg/mouse of either IgG control or anti-CD20 antibody (SA271G2;Biolegend). On day 0, mice were inoculated with 5x105 tumor cells (MC38-EpCAM). On day 7, tumors were measured, and mice were randomized into treatment groups with a mean tumor volume of ˜50 mm³ (n=10 mice/group). Mice receiving tumor targeted superantigen were treated with i.p. injections of 20 μg/mouse of tumor targeted superantigen (C215Fab-SEA) on days 7, 8, 9, 10, 14, 15, 16 and 17. The tumor targeted superantigen C215Fab-SEA is a fusion protein which includes a tumor-reactive mAb (C215Fab) and the bacterial superantigen staphylococcal enterotoxin A (SEA). C215Fab-SEA was used as a model tumor targeted superantigen instead of, e.g., naptumomab estafenatox, in order to facilitate in vivo murine experiments. Mice receiving anti-PD-L1 antibody were treated with injections of 100 μg/mouse of antibody (Mouse IgGle3, pdl1-mab15; Invivogen) on days 10 and 14. PBS was used as a control. Tumors were measured twice per week. The results are shown in FIGS. 3-5.

B-cell depletion in vivo was validated in spleens of mice receiving only anti-CD20 antibody. As shown in FIG. 3, in mice treated with anti-CD20 antibody, more than 95% of B-cells were depleted relative to control.

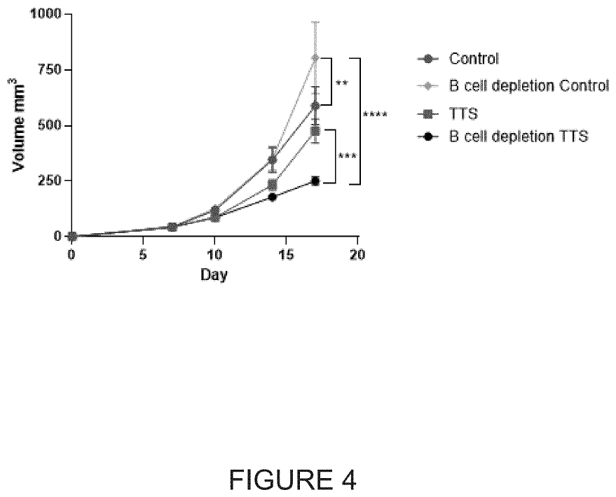

On day 17, at the completion of two treatment cycles, larger tumors were observed in mice treated with anti-CD20 antibody (i.e., depleted mice) relative to control (i.e., non-depleted mice) (**p=0.0013; FIG. 4).

Although treatment with anti-CD20 antibody alone significantly increased tumor volume, treatment with anti-CD20 antibody in combination with tumor targeted superantigen significantly decreased tumor volume (tumor growth inhibition (TGI)=67%; ****p<0.0001; FIG. 4). Moreover, tumor targeted superantigen treatment was significantly more effective in the depleted mice relative to the non-depleted mice (***p=0.0008 ; FIG. 4). The anti-tumor effect of tumor targeted superantigen in combination with anti-CD20 antibody was greater than the additive effect of each agent when administered alone.

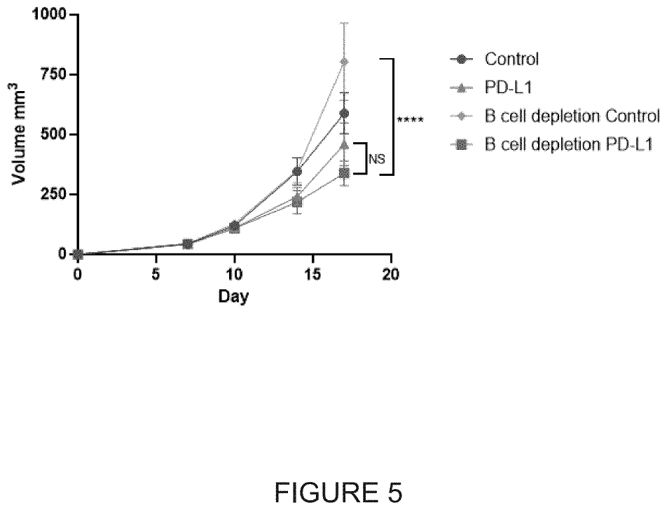

Treatment with anti-PD-L1 antibody also significantly decreased tumor volume in the depleted mice (****p<0.0001; FIG. 5). However, unlike the tumor targeted superantigen, anti-PD-L1 antibody was not significantly more effective in the depleted mice relative to the non-depleted mice (FIG. 5).

These results demonstrate the potential of tumor targeted superantigen (e.g., C215Fab-SEA or naptumomab estafenatox) combined with a B-cell depleting agent (e.g., an anti-CD20 antibody, e.g., obinutuzumab) for the treatment of cancer (e.g., colon cancer).

### Example 2: B-Cell Depletion Enhances Cytotoxic T-cell Expansion and Infiltration Following Tumor Targeted Superantigen Treatment

This Example describes an ex vivo study testing the effect of a tumor targeted superantigen in combination with a B-cell depleting agent (an anti-CD20 antibody) on cytotoxic T-cell (CD8+) expansion and infiltration into the tumor microenvironment (TME).

Mice were treated with either: (i) IgG control. (ii) anti-CD20 antibody, (iii) tumor targeted superantigen. (iv) anti-CD20 antibody and tumor targeted superantigen, (v) anti-PD-L1 antibody, or (vi) anti-CD20 antibody and anti-PD-L1 antibody. In brief, on day -7, mice were injected i.v. with 250 μg/mouse of either IgG control or anti-CD20 antibody (SA271G2;Biolegend). On day 0, mice were inoculated with 5×10⁵ tumor cells (MC38-EpCAM). On day 7, tumors were measured, and mice were randomized into treatment groups with a mean tumor volume of ˜50 mm³. Mice receiving tumor targeted superantigen were treated with 4 consecutive daily i.p. injections starting on day 7 of 20 μg/mouse of tumor targeted superantigen (C215Fab-SEA). Mice receiving anti-PD-L1 antibody were treated with one injection of 100 μg/mouse of antibody (Mouse IgG1e3, pdl1-mab15; Invivogen) on day 10. PBS was used as a control.

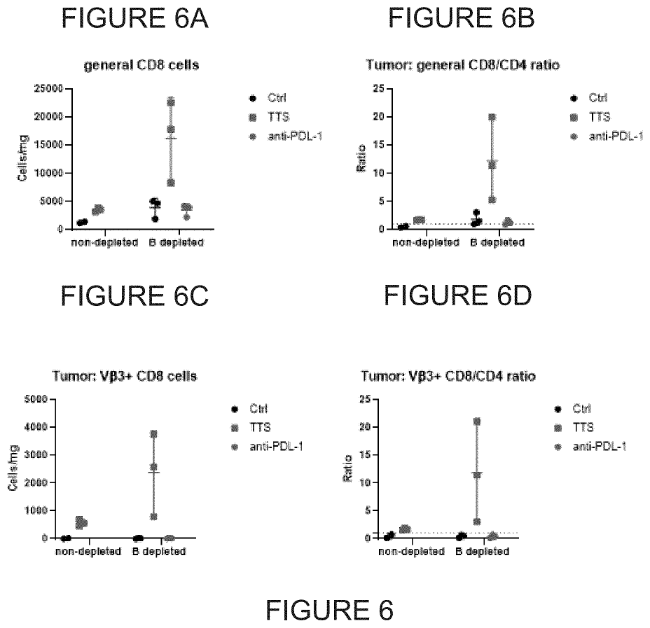

On day 13, mice from each group (n=3) were sacrificed, and tumors were removed and processed to a single cell suspension. The tumor cell suspension was analyzed by FACS to determine the number of CD8+ T-cells, Vβ3+CD8+ T-cells (a T-cell population known to be activated by tumor targeted superantigen treatment), and CD4+ T-cells. As can be seen in FIG. 6A and FIG. 6C, tumor infiltration of CD8+ T-cells and Vβ3+CD8+ T-cells was increased following treatment with tumor targeted superantigen alone (i.e., “non-depleted”) and treatment with tumor targeted superantigen in combination with anti-CD20 antibody (i.e., “depleted”). However greater infiltration of CD8+ T-cells and Vβ3+CD8+ T-cells was observed in tumors from the depleted mice relative to tumors from the non-depleted mice. In addition, the ratio of CD8:CD4 was significantly higher in tumors from the depleted mice relative to the non-depleted mice (FIG. 6B and FIG. 6D). No effect on cytotoxic T-cell infiltration was observed following anti-PD-L1 treatment (FIGS. 6A-6D).

Together, these results suggest that depletion of B-cells with a B-cell depleting agent (e.g., an anti-CD20 antibody, e.g., obinutuzumab) increases general and V133+cytotoxic T-cell (CD8+) expansion and infiltration into the tumor microenvironment (TME) following treatment with a tumor targeted superantigen (e.g., C215Fab-SEA or naptumomab estafenatox).

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A superantigen conjugate for use in treating cancer, said superantigen being covalently linked to a targeting moiety that binds a cancer antigen wherein said use is in combination with a B-cell depleting agent, and said B-cell depleting agent is administered to a subject in need before administration of the superantigen conjugate to the subject.

2. A superantigen conjugate for use in promoting infiltration of T-cells into a tumor, said superantigen being covalently linked to a targeting moiety that binds a cancer antigen expressed by cancerous cells, wherein said use is in combination with a B-cell depleting agent;
and said B-cell depleting agent is administered to a subject in need before administration of the superantigen conjugate to the subject.

3. The superantigen conjugate for use according to claim 2, wherein the T-cells are CD8+ T-cells.

4. A superantigen conjugate for use in increasing the ratio of CD8+ T-cells to CD4+ T-cells in a tumor, said superantigen being covalently linked to a targeting moiety that binds a cancer antigen expressed by cancerous cells, wherein said use is in combination with B-cell depleting agent and said B-cell depleting agent is administered to a subject in need before administration of the superantigen conjugate to the subject.

5. A superantigen conjugate for use in reducing infiltration of regulatory T-cells (Tregs) into a tumor, said superantigen being covalently linked to a targeting moiety that binds a cancer antigen expressed by cancerous cells, wherein said use is in combination with B-cell depleting agent and said B-cell depleting agent is administered to a subject in need before administration of the superantigen conjugate to the subject.

6. A superantigen conjugate for use in treating cancer, said superantigen being covalently linked to a targeting moiety that binds a cancer antigen expressed by cancerous cells, wherein said use is in combination with B-cell depleting agent and said B-cell depleting agent is administered to a subject in need before administration of the superantigen conjugate to the subject.

7. The superantigen conjugate for use according to claim 1 or 6, wherein the cancer comprises a tumor.

8. The superantigen conjugate for use according to claim 7, wherein said combination of the B-cell depleting agent and the superantigen conjugate promotes infiltration of T-cells into the tumor.

9. The superantigen conjugate for use according to claim 7 or 8, wherein said combination of the B-cell depleting agent and the superantigen conjugate increases the ratio of CD8+ T-cells to CD4+ T-cells in the tumor.

10. The superantigen conjugate for use according to any one of claims 7-9, wherein said combination of the B-cell depleting agent and the superantigen conjugate reduces infiltration of Tregs into the tumor.

11. The superantigen conjugate for use of any one according to claim 2-5 or 7-10, wherein the tumor is a solid tumor.

12. The superantigen conjugate for use according to any one of claims 1-11, wherein the superantigen comprises Staphylococcal enterotoxin A or an immunologically variant and/or fragment thereof.

13. The superantigen conjugate for use according to any one of claims 1-12, wherein the superantigen comprises the amino acid sequence of SEQ ID NO: 3, or an immunologically reactive variant and/or fragment thereof.

14. The superantigen conjugate for use according to any one of claims 1-13, wherein the cancer antigen is EpCAM or 5T4.

15. The superantigen conjugate for use according to claim 14, wherein the cancer antigen is 5 T4.

16. The superantigen conjugate for use according to any one of claims 1-15, wherein the targeting moiety is an antibody.

17. The superantigen conjugate for use according to claim 16, wherein the antibody is an anti-5T4 antibody or an anti-EpCAM antibody.

18. The superantigen conjugate for use according to claim 17, wherein the antibody is an anti-5T4 antibody.

19. The superantigen conjugate for use according to claim 18, wherein the anti-5T4 antibody comprises a Fab fragment that binds a 5T4 cancer antigen.

20. The superantigen conjugate for use according to claim 19, wherein the anti-5T4 antibody comprises a heavy chain comprising amino acid residues 1-222 of SEQ ID NO: 8 and a light chain comprising amino acid residues 1-214 of SEQ ID NO: 9.

21. The superantigen conjugate for use according to any one of claims 1-20, wherein the superantigen conjugate comprises a first protein chain comprising SEQ ID NO: 8 and a second protein chain comprising SEQ ID NO: 9.

22. The superantigen conjugate for use according to of any one of claims 1-21, wherein the B-cell depleting agent is an anti-CD20 antibody.

23. The superantigen conjugate for use according to claim 22, wherein the anti-CD20 antibody is selected from ibritumomab, obinutuzumab, ocaratuzumab, ocrelizumab, ofatumumab, rituximab, veltuzumab, tositumomab, ublituximab, veltuzumab, PRO131921, and TRU-015.

24. The superantigen conjugate for use according to claim 23, wherein the anti-CD20 antibody is obinutuzumab.

25. The superantigen conjugate for use according to any one of claims 1-24, wherein the cancer is a 5T4-expressing or an EpCAM-expressing cancer.

26. The superantigen conjugate for use according to any one of claim 25, wherein the cancer is a 5T4-expressing cancer.

27. The superantigen conjugate for use according to any one of claims 1-26, wherein the cancer is selected from breast cancer, bladder cancer, cervical cancer, colon cancer, colorectal cancer, endometrial cancer, gastric cancer, head and neck cancer, liver cancer, melanoma, mesothelioma, non-small cell lung cancer, ovarian cancer, pancreatic cancer, prostate cancer, renal cell cancer, skin cancer, and a hematopoietic cancer.

28. The superantigen conjugate for use according to any one of claims 1-27, wherein the method further comprises administering to the subject an immunopotentiator.

29. The superantigen conjugate for use according to any one of claim 28, wherein the immunopotentiator is a CTLA-4-based or a PD-1-based inhibitor.

30. The superantigen conjugate for use according to claim 29, wherein the PD-1-based inhibitor is a PD-1 or PD-L1 inhibitor.

31. The superantigen conjugate for use according to claim 30, wherein the PD-1 inhibitor is an anti-PD-1 antibody.

32. The superantigen conjugate for use according to claim 31, wherein the anti-PD-1 antibody is selected from nivolumab pembrolizumab, and cemiplimab.

33. The superantigen conjugate for use according to claim 30, wherein the PD-L1 inhibitor is an anti-PD-L1 antibody.

34. The superantigen conjugate for use according to 33, wherein the anti-PD-L1 antibody is selected from atezolizumab, avelumab, and durvalumab.

35. The superantigen conjugate for use according to any one of claims 1-27, wherein the method further comprises administering to the subject a chemotherapeutic agent.

36. The superantigen conjugate for use according to claim 35, wherein the chemotherapeutic agent is docetaxel.

37. A method of improving the efficacy of a superantigen conjugate comprising a superantigen covalently linked to a targeting moiety that binds a cancer antigen in treating cancer in a subject in need thereof, the method comprising administering to the subject an effective amount of a B-cell depleting agent prior to administering the superantigen conjugate to the subject.

38. A method of promoting infiltration of T-cells into a tumor in a subject in need thereof, the method comprising administering to the subject: (i) an effective amount of a B-cell depleting agent; and then (ii) an effective amount of a superantigen conjugate comprising a superantigen covalently linked to a targeting moiety that binds a cancer antigen expressed by cancerous cells of the tumor, thereby to promote infiltration of T-cells into the tumor.

39. The method of claim 38, wherein the T-cells are CD8+ T-cells.

40. A method of increasing the ratio of CD8+ T-cells to CD4+ T-cells in a tumor in a subject in need thereof, the method comprising administering to the subject: (i) an effective amount of a B-cell depleting agent; and then (ii) an effective amount of a superantigen conjugate comprising a superantigen covalently linked to a targeting moiety that binds a cancer antigen expressed by cancerous cells of the tumor, thereby to increase the ratio of CD8+ T-cells to CD4+ T-cells in the tumor.

41. A method of reducing infiltration of regulatory T-cells (Tregs) into a tumor in a subject in need thereof, the method comprising administering to the subject: (i) an effective amount of a B-cell depleting agent; and then (ii) an effective amount of a superantigen conjugate comprising a superantigen covalently linked to a targeting moiety that binds a cancer antigen expressed by cancerous cells of the tumor, thereby to reduce infiltration of Tregs into the tumor.

42. A method of treating cancer in a subject in need thereof, the method comprising administering to the subject: (i) an effective amount of a B-cell depleting agent; and then (ii) an effective amount of a superantigen conjugate comprising a superantigen covalently linked to a targeting moiety that binds a cancer antigen expressed by cancerous cells of the tumor.

43. The method of claim 38 or 42, wherein the cancer comprises a tumor.

44. The method of claim 43, wherein administration of the B-cell depleting agent and the superantigen conjugate to the subject promotes infiltration of T-cells into the tumor.

45. The method of claim 43 or 44, wherein administration of the B-cell depleting agent and the superantigen conjugate to the subject increases the ratio of CD8+ T-cells to CD4+ T-cells in the tumor.

46. The method of any one of claims 43-45, wherein administration of the B-cell depleting agent and the superantigen conjugate to the subject reduces infiltration of Tregs into the tumor.

47. The method of any one of claim 38-41 or 43-46, wherein the tumor is a solid tumor.

48. The method of any one of claims 38-47, wherein the superantigen comprises Staphylococcal enterotoxin A or an immunologically variant and/or fragment thereof.

49. The method of any one of claims 38-48, wherein the superantigen comprises the amino acid sequence of SEQ ID NO: 3, or an immunologically reactive variant and/or fragment thereof.

50. The method of any one of claims 38-49, wherein the cancer antigen is EpCAM or 5T4.

51. The method of claim 50, wherein the cancer antigen is 5T4.

52. The method of any one of claims 38-51, wherein the targeting moiety is an antibody.

53. The method of claim 52, wherein the antibody is an anti-5T4 antibody or an anti-EpCAM antibody.

54. The method of claim 53, wherein the antibody is an anti-5T4 antibody.

55. The method of claim 54, wherein the anti-5T4 antibody comprises a Fab fragment that binds a 5T4 cancer antigen.

56. The method of claim 55, wherein the anti-5T4 antibody comprises a heavy chain comprising amino acid residues 1-222 of SEQ ID NO: 8 and a light chain comprising amino acid residues 1-214 of SEQ ID NO: 9.

57. The method of any one of claims 38-56, wherein the superantigen conjugate comprises a first protein chain comprising SEQ ID NO: 8 and a second protein chain comprising SEQ ID NO: 9.

58. The method of any one of claims 38-57, wherein the B-cell depleting agent is an anti-CD20 antibody.

59. The method of claim 58, wherein the anti-CD20 antibody is selected from ibritumomab, obinutuzumab, ocaratuzumab, ocrelizumab, ofatumumab, rituximab, veltuzumab, tositumomab, ublituximab, veltuzumab, PRO131921, and TRU-015.

60. The method of claim 59, wherein the anti-CD20 antibody is obinutuzumab.

61. The method of any one of claims 38-60, wherein the cancer is a 5T4-expressing or an EpCAM-expressing cancer.

62. The method of any one of claim 61, wherein the cancer is a 5T4-expressing cancer.

63. The method of any one of claims 38-62, wherein the cancer is selected from breast cancer, bladder cancer, cervical cancer, colon cancer, colorectal cancer, endometrial cancer, gastric cancer, head and neck cancer, liver cancer, melanoma, mesothelioma, non-small cell lung cancer, ovarian cancer, pancreatic cancer, prostate cancer, renal cell cancer, skin cancer, and a hematopoietic cancer.

64. The method of any one of claims 38-63, wherein the method further comprises administering to the subject an immunopotentiator.

65. The method of any one of claim 64, wherein the immunopotentiator is a CTLA-4-based or a PD-1-based inhibitor.

66. The method of claim 65, wherein the PD-1-based inhibitor is a PD-1 or PD-L1 inhibitor.

67. The method of claim 66, wherein the PD-1 inhibitor is an anti-PD-1 antibody.

68. The method of claim 67, wherein the anti-PD-1 antibody is selected from nivolumab pembrolizumab, and cemiplimab.

69. The method of claim 66, wherein the PD-L1 inhibitor is an anti-PD-L1 antibody.

70. The method of claim 69, wherein the anti-PD-L1 antibody is selected from atezolizumab, avelumab, and durvalumab.

71. The method of any one of claims 38-70, wherein the method further comprises administering to the subject a chemotherapeutic agent.

72. The method of claim 71, wherein the chemotherapeutic agent is docetaxel.

73. A pharmaceutical composition comprising: (i) an effective amount of a superantigen conjugate comprising a superantigen covalently linked to a targeting moiety that binds a cancer antigen expressed by cancerous cells within the subject; (ii) an effective amount of a B-cell depleting agent; and (iii) a pharmaceutically acceptable excipient.

74. A kit comprising a first container containing a superantigen conjugate and a second container containing a B-cell depleting agent, and instructions for use of the superantigen conjugate in combination with the B-cell depleting agent according to any one of claims 1 to 37.
